(19) United States
(12) Patent Application Publication
(10) Pub. No.: US 2026/0035422 A1
(43) Pub. Date: Feb. 5, 2026

(54) FOLLISTATIN-LIKE 1 (FSTL1) FOR REPAIRING CARDIAC TISSUE

(71) Applicant: Regencor, Inc., San Carlos, CA (US)

(72) Inventors: Pilar RUIZ-LOZANO, San Carlos, CA (US); Anke KRETZ-ROMMEL, San Carlos, CA (US)

(21) Appl. No.: 19/257,266

(22) Filed: Jul. 1, 2025

Related U.S. Application Data
(63) Continuation of application No. PCT/US2025/035787, filed on Jun. 27, 2025.
(60) Provisional application No. 63/666,024, filed on Jun. 28, 2024.

(51) Int. Cl. C07K 14/47; A61K 38/00; A61L 27/22; A61L 27/52

(52) U.S. Cl. CPC C07K 14/473; A61L 27/227; A61L 27/52; A61K 38/00; A61L 2400/06; A61L 2430/20

(57) ABSTRACT

Provided herein are compositions comprising a glycoengineered follistatin-like (FSTL1) polypeptide with a distinctive glycosylation profile. Also provided herein are methods of using thereof for cardiac tissue repair and regeneration. Further provided herein are pharmaceutical compositions and kits comprising the glycoengineered FSTL1.

### RELATED APPLICATIONS

This application is a Continuation of International Application No. PCT/US2025/035787, filed on Jun. 27, 2025, which claims priority to U.S. Provisional Application Ser. No. 63/666,024, filed on Jun. 28, 2024, both of which are incorporated herein by reference in their entireties for all purposes.

### STATEMENT REGARDING FEDERALLY SPONSORED R&D

This invention was made with government support under Grant No. TRAN1-12907 awarded by the California Institute for Regenerative Medicine. The government has certain rights in the invention.

### INCORPORATION OF THE SEQUENCE LISTING

This application contains a Sequence Listing, which is incorporated herein by reference in its entirety. The accompanying Sequence Listing xml file, named “2025-06-26 Sequence Listing_ST26 048722-508001WO.xml” was created on Jun. 26, 2025 and is 18,829 bytes.

### BACKGROUND

Acute myocardial infarction (AMI) is a leading cause of death in the Western world, influenced by various environmental and genetic factors. The heart typically lacks the ability to naturally regenerate, making it susceptible to damage and left ventricular remodeling after a myocardial infarction or other ischemic events. This remodeling can lead to left ventricular dilatation and eventually heart failure. Immediately following a coronary occlusion, myocytes downstream from the occlusion undergo necrosis or apoptosis. Neutrophils infiltrate the affected tissue, followed by leukocytes, primarily macrophages, which help clear necrotic debris. Neutrophils can, however, harm surrounding myocytes by releasing reactive oxygen species and proteolytic enzymes. This damage results in the formation of a hypocellular scar, leading to impaired contractile function and eventual heart failure.

To reduce the significant impact of ischemic events like myocardial infarction, it is crucial to develop new approaches and formulations that can either support the survival of cardiomyocytes or stimulate their growth after injury. Therapies are needed to address and treat cardiac (myocardial) tissue following such injuries. Therefore, there is an urgent need for the development of innovative therapies and compositions to protect cardiomyocyte survival and/or promote the growth of cardiomyocytes following ischemic events, such as myocardial infarction. The present disclosure aims to mitigate the epidemiological challenges associated with AMI and related cardiac tissue damage.

### SUMMARY

Recognized herein is a need for the development of innovative therapies and compositions to protect cardiomyocyte survival and/or promote the growth of cardiomyocytes following ischemic events. The present disclosure describes, inter alia, compositions containing glycoengineered follistatin-like 1 (FSTL1) with a distinctive glycosylation profile, and methods of using the same for cardiac tissue repair and regeneration.

Provided herein is a composition including a glycoengineered follistatin-like 1 (FSTL1) polypeptide, wherein the glycoengineered FSTL1 polypeptide (i) includes O-linked glycosylation at one or more O-linked glycosylation-competent amino acid residues, and (ii) lacks N-linked glycosylation at one or more N-linked glycosylation-competent amino acid residues. In some embodiments, the amino acid sequence of the glycoengineered FSTL1 can include the polypeptide sequence of SEQ ID NO: 1, SEQ ID NO: 5, SEQ ID NO: 7, or SEQ ID NO: 8 or a fragment or variant of any thereof having at least 90% sequence identity to SEQ ID NO: 1, SEQ ID NO: 5, SEQ ID NO: 7, or SEQ ID NO: 8. In some embodiments, the amino acid sequence of the glycoengineered FSTL1 can include the polypeptide sequence of SEQ ID NO: 1, SEQ ID NO: 5, SEQ ID NO: 7, or SEQ ID NO: 8. In some embodiments, the one or more O-linked glycosylation-competent amino acid residues can be at (i) positions 107 and 284 in the polypeptide sequence of SEQ ID NO: 1; (ii) positions 106 and 283 in the polypeptide sequence of SEQ ID NO: 5, (iii) positions 87 and 264 in the polypeptide sequence of SEQ ID NO: 7, or (iv) positions 87 and 264 in the polypeptide sequence of SEQ ID NO: 8. In some embodiments, the one or more N-linked glycosylation-competent amino acid residues lacking N-link glycosylation can be at (i) positions 144, 175, and 180 in the polypeptide sequence of SEQ ID NO: 1; (ii) positions 143, 174, and 179 in the polypeptide sequence of SEQ ID NO: 5; (iii) positions 124, 155, and 160 in the polypeptide sequence of SEQ ID NO: 7; or (iv) positions 124, 155, and 160 in the polypeptide sequence of SEQ ID NO: 8.

In some embodiments, the glycoengineered FSTL1 polypeptide: (a) can (i) include O-linked glycosylation at positions 107 and 284 in the polypeptide sequence of SEQ ID NO: 1, and (ii) lacks N-linked glycosylation at positions 144, 175, and 180 in the polypeptide sequence of SEQ ID NO: 1; (b) can (i) include comprises O-linked glycosylation at positions 106 and 283 in the polypeptide sequence of SEQ ID NO: 5, and (ii) lack N-linked glycosylation at positions 143, 174, and 179 in the polypeptide sequence of SEQ ID NO: 5; (c) can (i) include O-linked glycosylation at positions 87 and 264 in the polypeptide sequence of SEQ ID NO: 7, and (ii) lack N-linked glycosylation at positions 124, 155, and 160 in the polypeptide sequence of SEQ ID NO: 7; or (d) can (i) include O-linked glycosylation at positions 87 and 264 in the polypeptide sequence of SEQ ID NO: 7, and (ii) lack N-linked glycosylation at positions 124, 155, and 160 in the polypeptide sequence of SEQ ID NO: 8.

In some embodiments, one or more N-linked glycosylation-competent amino acid residues in the polypeptide sequence of SEQ ID NO: 1 and SEQ ID NO: 7 can be substituted with one or more glycosylation-incompetent residues. In some embodiments, the glycoengineered FSTL1 can include the polypeptide sequence of SEQ ID NO: 5 or SEQ ID NO: 8, or a fragment or variant of any thereof having at least 90% sequence identity to SEQ ID NO: 5 or SEQ ID NO: 8. In some embodiments, the amino acid sequence of the glycoengineered FSTL1 can include the polypeptide sequence of SEQ ID NO: 5 or SEQ ID NO: 8. In some embodiments, the one or more N-linked glycosylation-competent amino acid residues lacking N-link glycosylation can be at (i) positions 143, 174, and 179 in the polypeptide sequence of SEQ ID NO: 5; or (ii) positions 124, 155, and 160 in the polypeptide sequence of SEQ ID NO: 8.

In some embodiments, the glycoengineered FSTL1 polypeptide: (a) can (i) include O-linked glycosylation at positions 106 and 283 in the polypeptide sequence of SEQ ID NO: 5, and (ii) lack N-linked glycosylation at positions 143, 174, and 179 in the polypeptide sequence of SEQ ID NO: 5; or (b) can (i) include O-linked glycosylation at positions 87 and 264 in the polypeptide sequence of SEQ ID NO: 8, and (ii) lack N-linked glycosylation at positions 124, 155, and 160 in the polypeptide sequence of SEQ ID NO: 8.

In some embodiments, the glycoengineered FSTL1 can repair cardiac tissue following an injury.

Also provided herein is a nucleic acid encoding a glycoengineered FSTL1 polypeptide, wherein the glycoengineered FSTL1 polypeptide (i) includes O-linked glycosylation at one or more O-linked glycosylation-competent amino acid residues, and (ii) lacks N-linked glycosylation at one or more N-linked glycosylation-competent amino acid residues.

In some embodiments, the nucleic acid sequence encoding the glycoengineered FSTL1 can include the nucleic acid sequence of SEQ ID NO: 2 or SEQ ID NO: 6, or a fragment or variant of any thereof having at least 90% sequence identity to SEQ ID NO: 2 or SEQ ID NO: 6. In some embodiments, the nucleic acid sequence of the glycoengineered FSTL1 can include the nucleic acid sequence of SEQ ID NO: 2 or SEQ ID NO: 6.

Also provided herein is a vector including the nucleic acid described herein.

Further provided herein is a method of repairing cardiac tissue following an injury in a subject in need thereof, the method including contacting the cardiac tissue with a composition including a glycoengineered FSTL1 polypeptide, wherein the glycoengineered FSTL1 polypeptide (i) includes O-linked glycosylation at one or more O-linked glycosylation-competent amino acid residues, and (ii) lacks N-linked glycosylation at one or more N-linked glycosylation-competent amino acid residues.

In some embodiments, the amino acid sequence of the glycoengineered FSTL1 can include the polypeptide sequence of SEQ ID NO: 1, SEQ ID NO: 5, SEQ ID NO: 7, or SEQ ID NO: 8 or a fragment or variant of any thereof having at least 90% sequence identity to SEQ ID NO: 1, SEQ ID NO: 5, SEQ ID NO: 7, or SEQ ID NO: 8. In some embodiments, the amino acid sequence of the glycoengineered FSTL1 can include the polypeptide sequence of SEQ ID NO: 1, SEQ ID NO: 5, SEQ ID NO: 7, or SEQ ID NO: 8. In some embodiments, the one or more O-linked glycosylation-competent amino acid residues can be at (i) positions 107 and 284 in the polypeptide sequence of SEQ ID NO: 1; or (ii) positions 87 and 264 in the polypeptide sequence of SEQ ID NO: 7. In some embodiments, the one or more N-linked glycosylation-competent amino acid residues lacking N-link glycosylation can be at (i) positions 107 and 284 in the polypeptide sequence of SEQ ID NO: 1; (ii) positions 106 and 283 in the polypeptide sequence of SEQ ID NO: 5, (iii) positions 87 and 264 in the polypeptide sequence of SEQ ID NO: 7, or (iv) positions 87 and 264 in the polypeptide sequence of SEQ ID NO: 8.

In some embodiments, the glycoengineered FSTL1 polypeptide: (a) can (i) include O-linked glycosylation at positions 107 and 284 in the polypeptide sequence of SEQ ID NO: 1, and (ii) lack N-linked glycosylation at positions 144, 175, and 180 in the polypeptide sequence of SEQ ID NO: 1; (b) can (i) include O-linked glycosylation at positions 106 and 283 in the polypeptide sequence of SEQ ID NO: 5, and (ii) lack N-linked glycosylation at positions 143, 174, and 179 in the polypeptide sequence of SEQ ID NO: 5; (c) can (i) include O-linked glycosylation at positions 87 and 264 in the polypeptide sequence of SEQ ID NO: 7, and (ii) lack N-linked glycosylation at positions 124, 155, and 160 in the polypeptide sequence of SEQ ID NO: 7; or (d) can (i) include O-linked glycosylation at positions 87 and 264 in the polypeptide sequence of SEQ ID NO: 7, and (ii) lacks N-linked glycosylation at positions 124, 155, and 160 in the polypeptide sequence of SEQ ID NO: 8.

In some embodiments, one or more N-linked glycosylation-competent amino acid residues in the polypeptide sequence of SEQ ID NO: 1 and SEQ ID NO: 7 can be substituted with one or more glycosylation-incompetent residues. In some embodiments, the glycoengineered FSTL1 can include the polypeptide sequence of SEQ ID NO: 5 or SEQ ID NO: 8, or a fragment or variant of any thereof having at least 90% sequence identity to SEQ ID NO: 5 or SEQ ID NO: 8. In some embodiments, the amino acid sequence of the glycoengineered FSTL1 can include the polypeptide sequence of SEQ ID NO: 5 or SEQ ID NO: 8. In some embodiments, the one or more N-linked glycosylation-competent amino acid residues lacking N-link glycosylation can be at (i) positions 143, 174, and 179 in the polypeptide sequence of SEQ ID NO: 5; or (ii) positions 124, 155, and 160 in the polypeptide sequence of SEQ ID NO: 8.

In some embodiments, the glycoengineered FSTL1 polypeptide: (a) can (i) include O-linked glycosylation at positions 106 and 283 in the polypeptide sequence of SEQ ID NO: 5, and (ii) lack N-linked glycosylation at positions 143, 174, and 179 in the polypeptide sequence of SEQ ID NO: 5; or (b) can (i) include O-linked glycosylation at positions 87 and 264 in the polypeptide sequence of SEQ ID NO: 8, and (ii) lack N-linked glycosylation at positions 124, 155, and 160 in the polypeptide sequence of SEQ ID NO: 8.

In some embodiments, the injury can be an ischemia reperfusion cardiac injury. In some embodiments, the injury can be a myocardial infarction. In some embodiments, the injury can be due to ischemic heart disease and/or a hypoplastic heart. In some embodiments, the repairing can result in increased recovery of the cardiac tissue. In some embodiments, the repairing can result in a reduction in fibrotic scar area in the cardiac tissue following the injury. In some embodiments, the repairing can result in an increase of perfused area in the cardiac tissue. In some embodiments, the repairing can result in an increase in cardiomyocyte cytokinesis in the cardiac tissue. In some embodiments, the cardiomyocyte cytokinesis can be determined by Aurora B kinase expression. In some embodiments, the repairing can result in an increased number of cardiomyocytes. In some embodiments, the repairing can result in an increased level of transcripts encoding cardiac-specific contractile proteins in the cardiomyocytes. In some embodiments, the cardiac-specific contractile proteins can be TNT-T2, myh6, mlc2v, and/or mlc2a. In some embodiments, the repairing can result in an increase of actinin⁺ cells with rhythmic contractile Ca²⁺ in cardiomyocytes. In some embodiments, the repairing can result in an increase of cardiomyocyte cell cycle entry. In some embodiments, the cardiomyocyte cell cycle entry can be assessed by expression of phosphor-histone H3. In some embodiments, the repairing can result in an increased vascularization in the area of the injury in the cardiac tissue. In some embodiments, the increased vascularization can be determined by expression of von Willebrand factor (vWF) or smooth muscle actin in blood vessel cells.

In some embodiments, the contacting can be immediately following the injury. In some embodiments, the contacting can be any time after the injury. In some embodiments, the composition can be delivered to the injury systemically. In some embodiments, the composition can be delivered to the injury endocardially. In some embodiments, the composition can be delivered to the injury epicardially. In some embodiments, the composition can be injected directly to the injury. In some embodiments, the composition can be delivered to the injury via a drug-eluting stent. In some embodiments, the composition can be delivered to the injury via a catheter. In some embodiments, the composition can be delivered to the injury via coronary infusion. In some embodiments, the composition can be delivered to the injury in a hydrogel. In some embodiments, the glycoengineered FSTL1 can be expressed at the injury by use of modified RNAs (modRNAs) or genomic editing.

Also provided herein is a sterile pharmaceutical composition including (a) the composition of the present disclosure, the nucleic acid of the present disclosure, or the vector of the present disclosure; and (b) one or more pharmaceutically acceptable excipients.

Also provided herein is a kit including (a) the composition of the present disclosure, the nucleic acid of the present disclosure, or the vector of the present disclosure; (b) one or more pharmaceutically acceptable excipients; and (c) an instruction for use of the kit.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a MS/MS spectra of GalNAc-Gal-Neu5Ac modified peptide SC: 83S-R102 of the glycoengineered FSTL1 (without signaling peptide). To confirm the composition of the O-glycans and to identify the O-glycosylation site, two enzyme digestion (Lys-C/trypsin and Lys-C), high-resolution tandem mass spectrometry with Electron-Activated Dissociation (EAD) fragmentation were applied to the O-glyco-peptide characterization

FIG. 2 is MS/MS spectra of GalNAc-Gal-2Neu5Ac modified peptide SC: 254G-K272 of the glycoengineered FSTL1 (without signal peptide) collected by EAD mode. To confirm the composition of the O-glycans and to identify the O-glycosylation site, two enzyme digestion (Lys-C/trypsin and Lys-C), high-resolution tandem mass spectrometry with Electron-Activated Dissociation (EAD) fragmentation were applied to the O-glyco-peptide characterization.

FIG. 3 is SDS-page gels for optimizing the signal peptide of glycoengineered FSTL1.

FIG. 4 is HPLC graphs for optimizing the signal peptide of glycoengineered FSTL1.

FIG. 5 is an SDS-PAGE of glycoengineered FSTL1, HEK FSTL1, and bacterial FSTL1.

FIG. 6 is a schematic summarizing the workflow involved in cloning and clone screening in Example 7.

### DETAILED DESCRIPTION

The present disclosure pertains to compositions, methods, and kits for cardiac tissue repair, specifically involving the utilization of follistatin-like 1 (FSTL1) polypeptides. The present disclosure describes, inter alia, the utilization of glycoengineered FSTL1 polypeptides with a distinct glycosylation profile, characterized by O-linked glycosylation and the absence of N-linked glycosylation, for cardiac tissue repair. Glycoengineered FSTL1 polypeptides described herein exhibit a unique glycosylation profile, featuring O-linked glycosylation while lacking N-linked glycosylation. The presently described compositions encompass glycoengineered FSTL1 polypeptides, which can be utilized for repairing cardiac tissue following injuries.

The present disclosure provides innovative solutions for addressing the challenges associated with cardiac tissue damage, including those arising from acute myocardial infarction and ischemic events. By employing glycoengineered FSTL1 polypeptides with specific glycosylation characteristics, the present disclosure aims to enhance the regenerative potential of these proteins, promoting cardiomyocyte survival and growth following cardiac injuries. Furthermore, the present disclosure encompasses the sterile pharmaceutical compositions containing the glycoengineered FSTL1 polypeptide with a unique glycosylation pattern. These compositions can be formulated with pharmaceutically acceptable excipients to ensure their safety and efficacy when administered to subjects in need of cardiac tissue repair.

### Definition

All terms are intended to be understood as they would be understood by a person skilled in the art. Unless defined otherwise, all technical and scientific terms used herein have the same meaning as commonly understood by one of ordinary skill in the art to which the disclosure pertains.

The term “about” or “approximately” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

The phrase “cardiac tissue,” as used herein, refers to any tissue of the heart. Cardiac tissue can include myocardial tissue, tissue of the epicardium, and tissue of the endocardium. Cardiac tissue can include any of the cell types found within the heart, such as, but not limited to, myocardial cells.

A “subject” or “individual” can be a vertebrate, a mammal, or a human. Mammals include, but are not limited to, farm animals, sport animals, pets, primates, mice and rats. In one aspect, a subject can be a human.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub combination was individually and explicitly disclosed herein.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Glycoengineered FSTL1

Provided herein are compositions containing glycoengineered follistatin-like 1 (FSTL1) polypeptide. FSTL1, as provided herein, is a glycoengineered FSTL1. In some embodiments, the glycoengineered FSTL1 polypeptide is e.g., a modified FSTL1 polypeptide, an engineered FSTL1 polypeptide, or a mutated FSTL1 polypeptide. In some embodiments, the compositions can include one or more pharmaceutically acceptable excipients or carriers, which are further explained infra.

Glycosylation refers to a post-translation modification process in which one or more carbohydrate moieties (e.g., glycans) can be enzymatically attached to a protein. Glycosylation can involve different types of sugar moieties and can occur in various cellular compartments. In proteins, the N-linked oligosaccharide can be attached to the amide group of an asparagine residue within the consensus peptide sequence NXS/T, where X can be any amino acid except proline. The most common O-glycosylations are O-acetylgalactosamine (O-GalNAc) and O-acetylglucosamine (O-GlcNAc). O-GalNAc can be attached to the hydroxyl group of the protein serine or threonine residues through an α-linkage, while O-GlcNAc can be attached through a β-linkage. Structurally, O-glycosylation can involve monosaccharides and/or oligosaccharide complexes of several different monosaccharides.

FSTL1 can be glycoengineered so that it is unable to be N-linked glycosylated at one or more N-linked glycosylation competent residues, while maintaining O-linked glycosylation at one or more O-linked glycosylation competent residues. For example, some or all N-linked glycosylation-competent asparagine amino acids in the FSTL1 polypeptide sequence can be substituted with a glycosylation-incompetent amino acid (for example, glutamine). In other embodiments, a glycoengineered FSTL1 can be engineered so that it is unable to be O-linked glycosylated at one or more O-linked glycosylation competent residues. For example, one or more O-linked glycosylation-competent serine, threonine, tyrosine, hydroxylysine, or hydroxyproline residues in the FSTL1 polypeptide sequence can be substituted with a glycosylation-incompetent amino acid (for example, alanine).

In some embodiments, a protein can be glycoengineered with a minimal number of carbohydrate moieties or completely lacking any carbohydrate moieties. In some embodiments, a protein can be glycoengineered in a way that completely lacks any carbohydrate modification whatsoever (for example, N-linked glycans, O-linked glycans, or phospho-glycans). In another embodiment, a protein can be glycoengineered to have a decreased carbohydrate modification relative to the amount of glycosylation that occurs in vivo under normal physiological conditions in mammalian cells. In yet other embodiments, a protein can be glycoengineered so that all or at least some of the glycosylation-competent amino acid residues (such as N-linked, O-linked, or phospho-glycan-competent amino acid residues) can be substituted with glycosylation-incompetent amino acid residues.

Methods of studying protein glycosylation are well known in the art. Staining or affinity-based methods can detect whether a protein is glycosylated or not. A basic, simple method to determine whether a protein is glycosylated is to resolve it on SDS-PAGE and to stain the gel for glycoproteins. Affinity-based methods, e.g., saccharide-binding protein methods, enzyme-based methods, or antibody-based methods, can be used to determine glycosylation types. For example, in the widely used lectin binding assay, a protein sample can be resolved on SDS-PAGE and transferred onto a nitrocellulose or polyvinylidene fluoride (PVDF) membrane. The membrane can then be incubated with a specific lectin and labeled with a group, such as digoxigenin (DIG) or biotin that can further bind to a secondary antibody or to avidin, respectively, which can be conjugated to an enzyme that can catalyze a color-producing reaction (with alkaline-phosphatase) or luminescence-producing reaction (with horseradish peroxidase). In another example, click chemistry, utilizing specific enzymes, can be used to label and isolate glycosylated proteins. Site-specific glycosylation can be determined by employing, for example, a combination of specific enzymatic proteolysis, fractionation of glycopeptides (by e.g., liquid chromatography or affinity chromatography), and glycopeptide analysis by mass-spectrometry analysis.

Accordingly, provided herein is a composition containing glycoengineered FSTL1 polypeptide, wherein the glycoengineered FSTL1 polypeptide (i) can include O-linked glycosylation at one or more O-linked glycosylation-competent amino acid residues, and (ii) can lack N-linked glycosylation at one or more N-linked glycosylation-competent amino acid residues.

O-glycosylation can exhibit variability, which can be precisely controlled through targeted mutagenesis of amino acids, for example. This control can be achieved by manipulating the amino acid sequences to either enhance or inhibit the addition of O-glycans. Additionally, O-glycosylation can be regulated by expressing the construct in cell lines that are deficient in specific glycosyltransferases and glycosidases. These enzymes can be pivotal in the processing and turnover of glycans, and their absence can allow for selective modification of glycosylation patterns. Furthermore, the variability of O-glycosylation can be influenced by utilizing cell lines that lack enzymes responsible for modifying glycans. These enzymes can include various transferases that add functional groups such as acetyl, methyl, phosphate, and sulfate, among others. By carefully selecting cell lines deficient in these modifying enzymes, specific glycosylation patterns can be achieved, enabling the precise engineering of glycoproteins with desired properties. Overall, the control of O-glycosylation through mutagenesis and the use of specialized cell lines can provide a versatile approach for glycoengineering.

In some embodiments, a glycoengineered FSTL1 can include the amino acid sequence of SEQ ID NO: 1, which is a 308-AA polypeptide with an N-terminal 20-AA signal peptide which can be removed to generate a 288-AA mature polypeptide. The amino acid sequence of the mature FSTL1 polypeptide is provided in SEQ ID NO: 7. In some embodiments, a glycoengineered FSTL1 can have the amino acid sequence of SEQ ID NO: 5, which is a 307-AA polypeptide with an N-terminal 19-AA signal peptide which can be removed to generate a 288-AA mature glycoengineered FSTL1 polypeptide (SEQ ID NO: 8). The amino acid sequence of the mature glycoengineered FSTL1 polypeptide is provided in SEQ ID NO: 8.

In some embodiments, a glycoengineered FSTL1 can be engineered so that it is unable to be N-linked glycosylated by substituting one or more N-linked glycosylation-competent amino acids with N-linked glycosylation-incompetent amino acids, while preserving O-linked glycosylation at one or more O-linked glycosylation competent residues. For example, one or more asparagine (N) residues located at positions 144, 175, and/or 180 in the amino acid sequence of SEQ ID NO: 1 or at positions 124, 155, and/or 160 in the amino acid sequence of SEQ ID NO: 7 can be substituted with a glycosylation-incompetent amino acid (such as, but not limited to, glutamine (Q)), while maintaining O-linked glycosylation at one or more serine or threonine residues located at positions 107 and/or 284 in the amino acid sequence of SEQ ID NO: 1 or at positions 87 or 264 in the amino acid sequence of SEQ ID NO: 7. In some embodiments, the glycoengineered FSTL1 comprises the polypeptide sequence of SEQ ID NO: 1 or SEQ ID NO: 7 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 1 or SEQ ID NO: 7. In some embodiments, the glycoengineered FSTL1 comprises the polypeptide sequence of SEQ ID NO: 1 or SEQ ID NO: 7.

In some embodiments, the glycoengineered FSTL1 comprises the polypeptide sequence of SEQ ID NO: 5 or SEQ ID NO: 8 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 5 or SEQ ID NO: 8. In some embodiments, the glycoengineered FSTL1 comprises the polypeptide sequence of SEQ ID NO: 5 or SEQ ID NO: 8.

In some embodiments, the glycoengineered FSTL1 comprises one or more O-linked glycosylation-competent amino acid residues at positions 107 and 284 in the polypeptide sequence of SEQ ID NO: 1 and lacks N-linked glycosylation at one or more N-linked glycosylation competent amino acid residues at positions 144, 175, and 180 in the polypeptide sequence of SEQ ID NO: 1.

In some embodiments, the glycoengineered FSTL1 comprises one or more O-linked glycosylation-competent amino acid residues at positions 87 and 264 in the polypeptide sequence of SEQ ID NO: 7 and lacks N-linked glycosylation at one or more N-linked glycosylation competent amino acid residues at positions 124, 155, and 160 in the polypeptide sequence of SEQ ID NO: 7.

In some embodiments, the glycoengineered FSTL1 comprises O-linked glycosylation-competent amino acid residues at positions 106 and 283 in the polypeptide sequence of SEQ ID NO: 5 and has N-linked glycosylation competent residues (e.g., asparagine) at positions 143, 174, and 179 in the polypeptide sequence of SEQ ID NO: 5 substituted with N-linked glycosylation incompetent residues (e.g., glutamine).

In some embodiments, the glycoengineered FSTL1 comprises O-linked glycosylation-competent amino acid residues at positions 87 and 264 in the polypeptide sequence of SEQ ID NO: 8 and has N-linked glycosylation competent residues (e.g., asparagine) at positions 124, 155, and 160 in the polypeptide sequence of SEQ ID NO: 8 substituted with N-linked glycosylation incompetent residues (e.g., glutamine).

In some embodiments, the amino acid sequence of the glycoengineered FSTL1 can include the polypeptide sequence of SEQ ID NO: 1 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 1. In some embodiments, the amino acid sequence of the glycoengineered FSTL1 can include the polypeptide sequence of SEQ ID NO: 1.

In some embodiments, the amino acid sequence of the glycoengineered FSTL1 can include the polypeptide sequence of SEQ ID NO: 5 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 5. In some embodiments, the amino acid sequence of the glycoengineered FSTL1 can include the polypeptide sequence of SEQ ID NO: 5.

In some embodiments, the amino acid sequence of the glycoengineered FSTL1 can include the polypeptide sequence of SEQ ID NO: 7 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 7. In some embodiments, the amino acid sequence of the glycoengineered FSTL1 can include the polypeptide sequence of SEQ ID NO: 7.

In some embodiments, the amino acid sequence of the glycoengineered FSTL1 can include the polypeptide sequence of SEQ ID NO: 8 or a fragment or variant thereof having at least 80%, at least 90%, at least 91%, at least 92%, at least 93%, at least 94%, at least 95%, at least 96%, at least 97%, at least 98%, or at least 99% sequence identity to SEQ ID NO: 8. In some embodiments, the amino acid sequence of the glycoengineered FSTL1 can include the polypeptide sequence of SEQ ID NO: 8.

In some embodiments, the glycoengineered FSTL1 of the present disclosure can repair cardiac tissue following an injury.

In some embodiments, the amino acid residue lacking N-linked glycosylation can be asparagine. In some embodiments, the amino acid residue having O-linked glycosylation can be serine or threonine.

### Production of Glycoengineered FSTL1

Glycoengineered FSTL1 can be obtained by producing FSTL1 in host cells that naturally do not post-translationally modify proteins with carbohydrate moieties or which have been engineered such that they are unable to post-translationally modify proteins with carbohydrate moieties at N-linked positions. A glycoengineered FSTL1 can be engineered such that it is unable to be N-linked glycosylated when produced using a eukaryotic or other glycosylation-competent host cell. In most biological contexts, glycosylation can be either N-linked or O-linked. The N-linked glycosylation process can occur in eukaryotes and widely in archaea, but very rarely in eubacteria. In N-linked glycosylation, glycans (i.e., carbohydrate-containing moieties) can be attached to the nitrogen atom of an asparagine amino acid side-chain. N-linked glycans can be almost always attached to the nitrogen atom of an asparagine (Asn) side chain that is present as a part of Asn-X-Ser/Thr consensus sequence, where X can be any amino acid except proline (Pro), serine (Ser), and threonine (Thr). O-linked glycosylation can be a form of glycosylation that can occur in the Golgi apparatus in eukaryotes. In O-linked glycosylation, glycans can be attached to the hydroxyl oxygen of serine, threonine, tyrosine, hydroxylysine, or hydroxyproline amino acid side-chains.

An engineered FSTL1 with a specific glycosylation pattern, for example, having O-linked glycosylation at one or more O-linked glycosylation-competent amino acid residues and lacking N-linked glycosylation at one or more N-linked glycosylation-competent amino acid residues, can be produced in host cells via transfection of a plasmid, viral vector carrying a nucleic acid encoding the FSTL1 or chemically synthetized mRNA or mRNA-mimetics. Alternatively, a nucleic acid encoding a glycoengineered FSTL1, which is further explained infra, with a specific glycosylation pattern, for example, having O-linked glycosylation at one or more O-linked glycosylation-competent amino acid residues and lacking N-linked glycosylation at one or more N-linked glycosylation-competent amino acid residues, can be integrated into a chromosome of the host cell under the control of an inducible or constitutively-expressing promoter. In yet another embodiment, a glycoengineered FSTL1 polypeptide with a specific glycosylation pattern, for example, having O-linked glycosylation at one or more O-linked glycosylation-competent amino acid residues and lacking N-linked glycosylation at one or more N-linked glycosylation-competent amino acid residues, can be produced in a host cell via delivery of modified mRNAs (modRNAs) encoding the glycoengineered FSTL1 polypeptide.

For example, a glycoengineered FSTL1 can be created by modifying the FSTL1 sequence (SEQ ID NO: 1) to replace asparagine residues at positions 144, 175, and 180 with glutamine, and subsequently optimizing the sequence for expression in e.g., CHO cells. The signal peptide can be optimized by testing for yield production, purity, cleavage efficiency, and O-glycosylation content in transiently transfected CHO cells. The optimized signal sequence can be synthesized, cloned into plasmid backbones, and transfected into e.g., CHO-K1 cells to generate stable cell lines. Glycoengineered FSTL1 can be produced in cultures, and the product can be purified by e.g., Ion Exchange and Cation Exchange Chromatography. The final product can be concentrated and analyzed for various quality parameters, including glycosylation patterns.

### Identification of O-Glycosylation Sites

The O-glycosylation sites on FSTL1 can be identified through e.g., peptide mapping, which can reveal multiple O-glycosylations annotated as protein with core-1 based O-glycans. The protein can then be subject to enzyme digestion and high-resolution tandem mass spectrometry (MS/MS) with electron-activated dissociation (EAD) fragmentation. The FSTL1 sample can be denatured with guanidine hydrochloride (Gdn-HCl) in Tris-HCl buffer, reduced by dithiothreitol (DTT), and cysteine-alkylated with iodoacetamide (IAM). After cleanup, the protein can be digested with Lys-C and Lys-C/trypsin sequentially to obtain peptides for analysis. The sample can be injected into an ExionLC coupled with a ZenoTOF 7600 mass spectrometer. Peptides can be separated using an Agilent/Poroshell SB-C18 column, and ultraviolet (UV) chromatograms can be set at 214 nm. The sample can then be analyzed in MS/MS mode with EAD fragmentation. The resulting mass spectra can be analyzed using the Protein Metrics Inc. (PMI)/Byonic search engine based on the theoretical FSTL1 sequences and the O-glycosylation database. The O-glyco-peptides identified can be further confirmed manually. The peak areas of extracted ion chromatograms (EICs) of O-glyco-peptides can be used to calculate the ratio of O-glycans. The identified O-glycosylation sites can be verified by EAD fragmentation, which can maintain labile modifications, allowing for the identification of glycosylation sites with modifications still intact. The identified O-glycosylation sites can be confirmed through comparison of MS/MS spectra with theoretical spectra, indicating the presence of core-1 based O-glycans.

### Nucleic Acids Encoding Glycoengineered FSTL1

Nucleic acids encoding glycoengineered FSTL1 polypeptide are provided herein and contemplated within the scope of the present disclosure. In some embodiments, a glycoengineered FSTL1 polypeptide can be encoded by the nucleic acid of SEQ ID NO: 2 and glycoengineered post-translationally. In some embodiments, a glycoengineered FSTL1 polypeptide can be encoded by the nucleic acid of SEQ ID NO: 6, which encodes a modified FSTL1 such that one or more N-linked glycosylation-competent amino acids are substituted with N-linked glycosylation-incompetent amino acids, while preserving O-linked glycosylation at one or more O-linked glycosylation competent residues.

The nucleic acid sequences can be codon optimized for optimal gene expression and translation efficiency in host cells. In some embodiments, the host cell can be CHO cells.

In some embodiments, an FSTL1 nucleic acid can be incorporated into a vector, such as an expression vector, using standard techniques known to one of skill in the art. Methods used to ligate the DNA construct including a nucleic acid of interest such as FSTL1, a promoter, a terminator, and other sequences and to insert them into a suitable vector are well known in the art. Additionally, vectors can be constructed using known recombination techniques (e.g., Invitrogen Life Technologies, Gateway Technology).

In some embodiments, it can be desirable to over-express FSTL1 nucleic acids at levels far higher than currently found in naturally-occurring cells. This result can be accomplished by the selective cloning of the nucleic acids encoding those polypeptides into multi-copy plasmids or placing those nucleic acids under a strong inducible or constitutive promoter. Methods for over-expressing desired polypeptides are common and well known in the art of molecular biology and examples can be found in Sambrook et al., Molecular Cloning: A Laboratory Manual, 2nd ed., Cold Spring Harbor, 2001.

A variety of host cells can be used to make a recombinant host cell that can express glycoengineered FSTL1. The host cell can be a cell that naturally produces FSTL1 or a cell that does not naturally produce FSTL1. For example, mammalian cells, such as, but not limited to, Chinese Hamster Ovary (CHO) cells or epicardium-derived cell cultures can be used to produce FSTL1. In some embodiments, the host cells can be, but not limited to, CHO cells, HEK293 cells, NS0 cells, BHK cells, Sf9 cells, Sf21 cells, COS cells, MDCK cells, PER.C6 cells, Jurkat cells, or HepG2 cells.

However, in other embodiments, cells derived from organisms that do not glycosylate proteins following translation (i.e., cells which do not post-translationally modify proteins with one or more carbohydrate moieties) can be used to produce glycoengineered FSTL1.

FSTL1-encoding nucleic acids or vectors containing them can be inserted into a host cell (e.g., CHO cell) using standard techniques for expression of the encoded FSTL1 polypeptide (e.g., the polypeptide of SEQ ID NO: 1, SEQ ID NO: 5, SEQ ID NO: 7, or SEQ ID NO: 8). Introduction of a DNA construct or vector into a host cell can be performed using techniques such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. General transformation techniques are well known in the art (see, e.g., Current Protocols in Molecular Biology (F. M. Ausubel et al. (eds) Chapter 9, 1987; Sambrook et al., Molecular Cloning: A Laboratory Manual, 3rd ed., Cold Spring Harbor, 2001; and Campbell et al., Curr Genet, 16:53-56, 1989). The introduced nucleic acids can be integrated into chromosomal DNA of the host cell or maintained as extrachromosomal replicating sequences. In yet another embodiment, an FSTL1 polypeptide can be produced in a host cell via delivery of chemically modified mRNAs encoding the mutated FSTL1 glycosylation-deficient polypeptide. See Modified mRNA directs the fate of heart progenitor cells and induces vascular regeneration after myocardial infarction. Zangi, et al. Nat Biotechnol. 31(10):898-907 (2013). Chemically modified RNAs, also referred to herein as modRNAs, can include, for example, modifications of phosphate into phosphorothioate internucleotidic linkages, modifications of the 2′-hydroxyl group of ribose, or other modifications to the phosphate backbone or sugar moieties of mRNA.

### Kits

Also provided herein are kits comprising (i) the glycoengineered FSTL1 of the present disclosure; and (ii) one or more pharmaceutically acceptable excipients. One or both of these kit components can be made to be sterile so that it can be administered to an individual in need (e.g., an individual with cardiac injury, such as MI). The kits can optionally contain a hydrogel (such as any of these disclosed herein) that can be seeded or infused with the glycoengineered FSTL1 of the present disclosure prior to administration to a subject. Alternatively, a pre-seeded or pre-infused hydrogel can be included in the kit along with written instructions regarding its use and application to injured cardiac (e.g., myocardial) tissue or the epicardium of a subject in need thereof. The kit can further comprise means for adhering the hydrogel to the epicardium or to injured cardiac (e.g., myocardial) tissue such as, without limitation, suturing material.

Any of the kits disclosed herein can also include a hydrogel (such as a self-polymerizing hydrogel) as a carrier for the glycoengineered FSTL1 of the present disclosure. In one embodiment, the kits can also include one or more catheters for delivery of the hydrogel (such as a hydrogel infused with a glycoengineered FSTL1 polypeptide) to the endocardium, epicardium, and/or one or more damaged areas of the myocardium.

The kit can also include written instructions for using the kit, such as instructions for infusing the glycoengineered FSTL1 of the present disclosure into a hydrogel, suturing the hydrogel to the myocardium or epicardium, infusing an epicardial-derived paracrine factor into a hydrogel (such as a self-polymerizing hydrogel) as well as delivery of the hydrogel to the epicardium or one or more damaged areas of the myocardium via catheter technology.

### Pharmaceutical Compositions

The presently described disclosure contemplates the glycoengineered FSTL1 of the present disclosure incorporated into a pharmaceutical composition (e.g., a sterile pharmaceutical composition) containing one or more pharmaceutically acceptable carriers.

A pharmaceutically acceptable carrier or a pharmaceutically acceptable excipient according to the present disclosure can be a component such as a carrier, diluent, or excipient of a composition that is compatible with the other ingredients of the composition in that it can be combined with the agents and/or compositions of the present invention without eliminating the biological activity of the agents or the compositions (for example, the glycoengineered FSTL1 of the present disclosure), and is suitable for use in subjects as provided herein without undue adverse side effects (such as toxicity, irritation, allergic response, and death). Side effects can be undue when their risk outweighs the benefit provided by the pharmaceutical composition. Non-limiting examples of pharmaceutically acceptable components include, without limitation, any of the standard pharmaceutical carriers such as phosphate buffered saline solutions, water, sterile water, polyethylene glycol, polyvinyl pyrrolidone, lecithin, arachis oil, sesame oil, emulsions such as oil/water emulsions or water/oil emulsions, microemulsions, nanocarriers and various types of wetting agents. Additives such as water, alcohols, oils, glycols, preservatives, flavoring agents, coloring agents, suspending agents, and the like can also be included in the composition along with the carrier, diluent, or excipient. In one embodiment, a pharmaceutically acceptable carrier appropriate for use in the compositions disclosed herein can be sterile, pathogen free, and/or otherwise safe for administration to a subject without risk of associated infection and other undue adverse side effects.

Any of the glycoengineered FSTL1-containing pharmaceutical compositions disclosed herein can be formulated for administration using any number of administrative methods available in the art. Administration can be by a variety of routes including patch, catheter, stent, oral, rectal, transdermal, subcutaneous, intravenous, intramuscular, intranasal, and the like. In some embodiments, any of the supra methods of administration, can be used for delivery of suspensions containing the glycoengineered FSTL1 of the present disclosure. These compositions can be effective as both injectable and oral compositions. Such compositions can be prepared in a manner well known in the pharmaceutical art and comprise at least one active compound. When employed as oral compositions, the polypeptide compositions can be protected from acid digestion in the stomach by a pharmaceutically acceptable protectant.

### METHODS OF THE DISCLOSURE

Provided herein are methods for repairing cardiac tissue injuries arising from e.g., myocardial infarction and ischemic events. In some embodiments, the injury can be an ischemia reperfusion cardiac injury. Reperfusion comprises restoration of blood flow or supply to the heart or cardiac (e.g., myocardial) tissue that has become ischemic or hypoxic. Modalities for reperfusion can include, but are not limited to, chemical dissolution of the occluding thrombus, i.e., thrombolysis, administration of vasodilators, angioplasty, percutaneous coronary intervention (PCI), catheterization and coronary artery bypass graft (CABG) surgery. In some embodiments, the injury can be a myocardial infarction. In some embodiments, the injury can be due to ischemic heart disease and/or a hypoplastic heart. Myocardial infarction (MI) can include a development of myocardial necrosis, which can be caused by the interruption of blood supply to the heart resulting in a critical imbalance between oxygen supply and demand of the myocardium. This can result from plaque rupture with thrombus formation in a coronary vessel leading to an acute reduction of blood supply to a portion of the myocardium; that is, an occlusion or blockage of a coronary artery following the rupture of a susceptible atherosclerotic plaque. If untreated for a sufficient period of time, the resulting ischemia or restriction in blood supply and oxygen shortage can cause damage or death, i.e., infarction of the heart. In general, this damage can be largely irreversible, and clinical therapies thus far mainly aim at delaying the progression of heart failure to prolong survival. Myocardial infarction can be assessed using clinical parameters and/or assessments known to those skilled in the art of diagnosing and/or treating the same, for example, physical examinations, detection of signs and symptoms of myocardial infarction, electrocardiogram, echocardiogram, chest X-ray, blood tests to detect cardiac biomarkers including troponins, CK, and CK-MB, etc.

The methods provided herein employs glycoengineered FSTL1 with specific glycosylation pattern to promote cardiomyocyte survival and growth following cardiac injuries. The glycoengineered FSTL1 of the present disclosure can include O-linked glycosylation at one or more O-linked glycosylation-competent amino acid residues, while lacking N-linked glycosylation at one or more N-linked glycosylation-competent amino acid residues.

In some embodiments, in any of the methods described herein, the amino acid sequence of the glycoengineered FSTL1 can include the polypeptide sequence of SEQ ID NO: 1 or a fragment or variant thereof having at least 80%, at least 90%, at least 91%, at least 92%, at least 93%, at least 94%, at least 95%, at least 96%, at least 97%, at least 98%, at least 99%, or 100% sequence identity to SEQ ID NO: 1, wherein one or more N-linked glycosylation competent amino acid residues at positions 144, 175, and/or 180 lack N-linked glycosylation, while maintaining one or more O-linked glycosylation at O-linked glycosylation competent residues at positions 107 and/or 284.

In some embodiments, in any of the methods described herein, the amino acid sequence of the glycoengineered FSTL1 can include the polypeptide sequence of SEQ ID NO: 7 or a fragment or variant thereof having at least 80%, at least 90%, at least 91%, at least 92%, at least 93%, at least 94%, at least 95%, at least 96%, at least 97%, at least 98%, at least 99%, or 100% sequence identity to SEQ ID NO: 7, wherein one or more N-linked glycosylation competent amino acid residues at positions 124, 155, and 180 lack N-linked glycosylation, while maintaining one or more O-linked glycosylation at O-linked glycosylation competent residues at positions 87 and/or 264.

In some embodiments, in any of the methods described herein, the amino acid sequence of the glycoengineered FSTL1 can include the polypeptide sequence of SEQ ID NO: 5 or a fragment or variant thereof having at least 80%, at least 90%, at least 91%, at least 92%, at least 93%, at least 94%, at least 95%, at least 96%, at least 97%, at least 98%, at least 99%, or 100% sequence identity to SEQ ID NO: 5, wherein one or more N-linked glycosylation competent amino acid residues at positions 143, 174, and 179 can be replaced with N-linked incompetent amino acid residue, while maintaining one or more O-linked glycosylation at O-linked glycosylation competent residues at positions 106 and/or 283.

In some embodiments, in any of the methods described herein, the amino acid sequence of the glycoengineered FSTL1 can include the polypeptide sequence of SEQ ID NO: 8 or a fragment or variant thereof having at least 80%, at least 90%, at least 91%, at least 92%, at least 93%, at least 94%, at least 95%, at least 96%, at least 97%, at least 98%, at least 99%, or 100% sequence identity to SEQ ID NO: 8, wherein one or more N-linked glycosylation competent amino acid residues at positions 124, 155, and 160 can be replaced with N-linked incompetent amino acid residue, while maintaining one or more O-linked glycosylation at O-linked glycosylation competent residues at positions 87 and/or 264.

In some embodiments, the amino acid residues at positions 107 and/or 284 of SEQ ID NO: 1, at positions 106 and/or 283 of SEQ ID NO: 5, at positions 87 and/or 264 of SEQ ID NO: 7, or at positions 87 and/or 264 of SEQ ID NO: 8 can be substituted with modified amino acids that are glyco-mimetics, such as, for example glycol amino acids.

### Delivery of Glycoengineered FSTL1

In any of the methods described herein, the contacting of the glycoengineered FSTL1 of the present disclosure can be immediately following the injury. In some embodiments, the contacting can be any time after the injury. In some embodiments, the composition can be delivered to the injury systemically. In some embodiments, the composition can be delivered to the injury endocardially. In some embodiments, the composition can be delivered to the injury epicardially. In some embodiments, the composition can be injected directly to the injury. In some embodiments, the effect on the cardiac (e.g., myocardial) tissue can be achieved with placement of the glycoengineered FSTL1 of the present disclosure systemically, using approved formulation. Systemic delivery can be achieved through various administration routes, including, but not limited to, intravenous (IV) infusion, intraperitoneal (IP) injection, intramuscular (IM) injection, or subcutaneous (SC) injection. In some embodiments, systemic delivery can be achieved through inhalation. In some embodiments, the composition can be delivered to the injury via a drug-eluting stent. In some embodiments, the composition can be delivered to the injury via a catheter. In some embodiments, the composition can be delivered to the injury via coronary infusion. In some embodiments, the composition can be delivered to the injury in a hydrogel. In some embodiments, the glycoengineered FSTL1 of the present disclosure can be expressed at the injury by use of modifiedRNAs (modRNAs) or genomic editing.

In some embodiments of any of the methods disclosed herein, the glycoengineered FSTL1 of the present disclosure can be infused, seeded, or embedded into a hydrogel. The hydrogel can then be contacted directly to the epicardium or an injured area of myocardium (such as an area of the myocardium exposed to an ischemic event, such as myocardial infarction). The hydrogel can then be applied to the epicardium or myocardium via suturing or by any other means known in the art for contacting the patch to the injured tissue. In yet other embodiments, the glycoengineered FSTL1 of the present disclosure can be a component of a hydrogel that can be delivered to the epicardium, to the endocardium, or to an injured area of myocardium (by, for example, catheter technology; Koudstaal et al., J. of Cardiovasc. Trans. Res. (2014) 7:232-241).

In some embodiments, a hydrogel can be selected from, but not limited to, polyacrylamide, polyethylene glycol (PEG), polyvinyl alcohol (PVA), sodium polyacrylate, alginate, chitosan, hydroxyethyl methacrylate (HEMA), poly(N-isopropylacrylamide) (PNIPAAm), agarose, gelatin, hyaluronic acid (HA), collagen, polyHEMA, pectinpoly(vinyl pyrrolidone) (PVP), carrageenan, sodium carboxymethylcellulose (CMC), and polyurethane.

Another option for delivery of the glycoengineered FSTL1 of the present disclosure to cardiac tissue can be as a component of a self-polymerizing hydrogel delivered by catheter technology. Further information related to this type of delivery can be found in Koudstaal et al., J. of Cardiovasc. Trans. Res. (2014) 7:232-241. Catheter delivery can also be employed for suspensions comprising glycoengineered, e.g., the glycoengineered FSTL1 mixed with gelfoam particles. Gelfoam particles are sterile, absorbable, and biodegradable gelatin sponge, which can be made from purified porcine gelatin. In some embodiments, gelfoam particles can serve as a carrier for the glycoengineered of the present disclosure. In some embodiments, gelfoam particles can be impregnated with the glycoengineered FSTL1 and can be placed at the target site, such as an injured or damaged heart tissue region.

In some other embodiments, the effect on the cardiac (e.g., myocardial) tissue can be achieved with placement of the glycoengineered FSTL1 of the present disclosure in proximity to the endocardial compartment (i.e., the endocardium), for example, by delivery via catheter technology. In some embodiments a suitable catheter may be a NOGA catheter (Johnson & Johnson). Catheters are thin, flexible tubes used for various medical procedures. One of the significant advantages of catheter-based delivery is that it is minimally invasive. Catheter-based delivery can allow for precise targeting. The glycoengineered FSTL1 of the present disclosure can be delivered directly to the specific area of the damaged cardiac tissue, minimizing potential side effects on healthy tissues. In many cases, catheter-based procedures can be guided by advanced imaging techniques, such as fluoroscopy, angiography, or intravascular ultrasound. In some embodiments, the catheter can be selected from, but not limited to, central venous catheter from e.g., Arrow®, Cordis®, or CVC Plus™, pulmonary artery catheter from e.g., Edwards Lifesciences™ or Cook Medical, angiographic catheter from e.g., Cordis™ and Cook Medical, balloon catheter from e.g., Boston Scientific™ or Medtronic™, percutaneous transluminal coronary angioplasty (PTCA) catheter from e.g., Boston Scientific™ or Medtronic™, atherectomy catheter from e.g., Spectranetics™ and Philips™.

In some embodiments, the effect on the cardiac (e.g., myocardial) tissue can be achieved with placement of the glycoengineered FSTL1 of the present disclosure endocardially into the heart by percutaneous catheter delivery systems, for example as the systems available developed by BioCardia (www.biocardia.com). Endocardial delivery can allow for a highly targeted approach, focusing on the inner lining of the heart, where it can have a direct impact on the cardiac tissue, including the endocardium itself and the underlying myocardium. The delivery of glycoengineered FSTL1 of the present disclosure to the endocardium can be achieved through minimally invasive procedures, such as cardiac catheterization. In this procedure, a catheter can be guided through a blood vessel, often starting from the femoral artery or vein, and advanced to the heart chambers. This can minimize the need for open-heart surgery. In some embodiments, the endocardial delivery can be guided by imaging techniques like fluoroscopy or endocardiography.

In some embodiments, the effect on the cardiac (e.g., myocardial) tissue can be achieved with placement of the glycoengineered FSTL1 of the present disclosure epicardially into the heart using catheter devices similar to those used in other applications (for example Epicardial Catheter System™, St. Jude Medical). Epicardial delivery of glycoengineered FSTL1 of the present disclosure can involve administering glycoengineered FSTL1 directly onto the epicardium, which is the outer layer of the heart. Epicardial delivery can be achieved through e.g., open-heart surgery or minimally invasive surgical techniques. Epicardial delivery can allow for direct contact between glycoengineered FSTL1 and the heart outer surface. This proximity can be advantageous as it can enable localized and concentrated administration of the glycoengineered FSTL1. Various known techniques can be employed for epicardial delivery, including, but not limited to, the direct application of glycoengineered FSTL1 using solutions or gels. In some cases, it can involve the use of specialized delivery devices. In some embodiments, epicardial delivery can be often guided by imaging techniques, such as echocardiography, to ensure precise placement and administration.

In some embodiments, the effect on the cardiac (e.g., myocardial) tissue can be achieved with placement of the glycoengineered FSTL1 of the present disclosure when impregnated in drug-diluting stents (for example, those available from Abbott Laboratories or Biosensors International, among others). Drug-eluting stents can be small, mesh-like devices typically made of metal, such as, but not limited to, stainless steel or cobalt-chromium, which can be inserted into narrowed or blocked coronary arteries during e.g., percutaneous coronary intervention (PCI) or angioplasty. The drug-eluting stent can be specially designed to have a coating that contains the glycoengineered FSTL1 of the present disclosure. In some embodiments, the glycoengineered FSTL1-containing coating can contain a biodegradable polymer that can release the glycoengineered FSTL1 over time, enabling controlled and sustained release at the site of stent implantation. In some embodiments, the biodegradable polymer can be selected from, but not limited to, poly(lactic-co-glycolic acid) (PLGA), poly(lactic acid) (PLA), poly(caprolactone) (PCL), poly(ester urethane), poly(anhydride), poly(carbonate), poly(orthoester), poly(polyol sebacate urethane) (PPSU), and poly(vinyl alcohol).

In some embodiments, the effect on the cardiac (e.g., myocardial) tissue can be achieved with placement of the glycoengineered FSTL1 of the present disclosure can be achieved by the use of compound or drugs that inhibit the glycosylation of the endogenous glycosylated FSTL1 protein, which is readily available and known to one of skill in the art.

In some embodiments, the effect on the cardiac (e.g., myocardial) tissue can be achieved with placement of the glycoengineered FSTL1 of the present disclosure can be achieved by introduction of modRNAs encoding for specific mutagenesis targeting N-glycosylation sites in the FSTL1 mRNA sequence. In some embodiments, synthetic RNA sequences can be engineered to encode the glycoengineered FSTL1 of the present disclosure. Various known transfection and delivery techniques, e.g., by use of lipid nanoparticles (LNPs) or viral vectors, can be utilized to enable the introduction of the modified RNA into the target cells. LMP refers to a lipid-nucleic acid particle or a nucleic acid-lipid particle (e.g., a stable nucleic acid-lipid particle). A LNP represents a particle made from lipids (e.g., a cationic lipid, a non-cationic lipid, and a conjugated lipid that prevents aggregation of the particle), and a nucleic acid, wherein the nucleic acid (e.g., small interfering RNA (siRNA), asymmetrical interfering RNA (aiRNA), microRNA (miRNA), single-stranded DNA (ssDNA), double-stranded DNA (dsDNA), single-stranded RNA (ssRNA), short hairpin RNA (shRNA), double-stranded RNA (dsRNA), messenger RNA (mRNA), self-amplifying RNA (saRNA), or a plasmid, including plasmids from which an interfering RNA or mRNA can be transcribed) encoding the glycoengineered FSTL1 of the present disclosure can be encapsulated within the lipid. LNPs typically can contain a cationic lipid, a non-cationic lipid, and a lipid conjugate (e.g., a PEG-lipid conjugate). Any of a variety of cationic lipids can be used in the LNP formulation, either alone or in combination with one or more other cationic lipid species or non-cationic lipid species.

In some embodiments, the effect on the cardiac (e.g., myocardial) tissue can be achieved with placement of the glycoengineered FSTL1 of the present disclosure can be achieved by genome editing using CRISPR-Cas9, TALENs, or other genome editing technology (see for example Yin, et al. Nat Biotechnol 32: 551-553 (2014)).

In some embodiments, the glycoengineered FSTL1 of the present disclosure can be delivered via genome editing using CRISPR-Cas system. In general, CRISPR system refers collectively to transcripts and other elements involved in the expression of or directing the activity of CRISPR-associated (Cas) genes, including sequences encoding a Cas gene, a tracr (trans-activating CRISPR) sequence (e.g., tracrRNA or an active partial tracrRNA), a tracr-mate sequence (encompassing a direct repeat and a tracrRNA-processed partial direct repeat in the context of an endogenous CRISPR system), a guide sequence (also referred to as a spacer in the context of an endogenous CRISPR system), or other sequences and transcripts from a CRISPR locus. In some embodiments, one or more elements of a CRISPR system can be derived from a type I, type II, or type III CRISPR system. In some embodiments, one or more elements of a CRISPR system can be derived from a particular organism comprising an endogenous CRISPR system, such as Streptococcus pyogenes. In general, a CRISPR system can be characterized by elements that promote the formation of a CRISPR complex at the site of a target sequence (also referred to as a protospacer in the context of an endogenous CRISPR system). In the context of formation of a CRISPR complex, target sequence refers to a sequence to which a guide sequence can be designed to have complementarity, where hybridization between a target sequence and a guide sequence can promote the formation of a CRISPR complex. A target sequence can comprise any polynucleotide, such as DNA or RNA polynucleotides. In some embodiments, a target sequence can be located in the nucleus or cytoplasm of a cell in the cardiac tissue. A sequence or template that can be used for recombination into the targeted locus comprising the target sequences can be referred to as an editing template.

In some embodiments, the glycoengineered FSTL1 of the present disclosure can be delivered via genome editing using transcription activator-like effector nucleases (TALENs) system. TALENs are a class of engineered proteins that have been developed for genome editing. TALENs can be used to specifically target and modify DNA sequences within the genome. TALENs consist of two main components: transcription activator-like effectors (TALEs) and a nuclease domain. In some embodiments, TALEs can be derived from bacteria and naturally function as transcription factors. They have a modular structure, with repeating amino acid motifs that correspond to individual DNA base pairs. These TALEs are customized to recognize specific DNA sequences, such as FSTL1 sequence. This specificity allows for precise targeting of genes for modification. TALENs can be coupled with a nuclease domain, typically the FokI endonuclease. When TALENs bind to their target DNA sequence, they bring the nuclease domain into close proximity, which results in the creation of double-strand breaks in the DNA, activating the cellular DNA repair machinery. This repair process can then be utilized to introduce specific changes to the DNA sequence. By providing a custom DNA template, such as DNA encoding the FSTL1 of the present disclosure, the gene can be inserted, deleted, or replaced with specific sequence.

The injured cardiac (e.g., myocardial) tissue can be contacted with any of the glycoengineered FSTL1 of the present disclosure compositions (such as pharmaceutical compositions) disclosed herein before, during, or subsequent to the injury to the cardiac (e.g., myocardial) tissue. In some embodiments, the cardiac (e.g., myocardial) tissue can be contacted with the glycoengineered FSTL1 of the present disclosure in a subject deemed at risk for cardiovascular disease, MI, or another myocardial ischemic event in order to mitigate or prevent injury to the myocardium by the event.

In some embodiments, the composition comprising the glycoengineered FSTL1 of the present disclosure can be delivered to the injury via coronary infusion. Coronary infusion, also known as intracoronary infusion, is a medical procedure involving the direct introduction of a therapeutic agent, such as the glycoengineered FSTL1 of the present disclosure, contrast agents, or other therapeutic agents into the coronary arteries, which supply blood to the myocardium. This procedure is typically performed during cardiac catheterization, a minimally invasive technique used for diagnosing and treating various cardiac conditions. A catheter can be guided through a blood vessel, often starting from the femoral artery or other access point, to reach the coronary arteries. This can minimize the need for open-heart surgery. Cardiac imaging techniques, such as fluoroscopy, can be used during coronary infusion procedures to ensure precise placement of the catheter and accurate delivery of the glycoengineered FSTL1.

In other embodiments, the cardiac (e.g., myocardial) tissue can be contacted with the glycoengineered FSTL1 of the present disclosure immediately following the onset of an ischemic event caused by cardiovascular disease or MI, such as about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 45 minutes, 1 hour, 1.5 hours, 2 hours, 2.5 hours, 3 hours, 3.5 hours, 4 hours, 4.5 hours, 5 hours 5.5 hours, 6 hours, 6.5 hours, 7 hours, 7.5 hours, 8 hours, 8.5 hours, 9 hours, 9.5 hours, 10 hours, 10.5 hours, 11 hours, 11.5 hours, or 12 hours or more (inclusive of all time periods falling in between these values). In some embodiments, the composition can be administered less than 1 minute after the cardiac injury.

Alternatively, in other embodiments, the cardiac (e.g., myocardial) tissue can be contacted with the glycoengineered FSTL1 of the present disclosure subsequent to the injury, such as at least 12 hours, 13 hours, 14 hours, 15 hours, 16 hours, 17 hours, 18 hours, 19 hours, 20 hours, 21 hours, 22 hours, 23 hours, 24 hours or 1 day, 2 days, 3 days, 4 days, 5 days, 6 days, 7 days, 8 days, 9 days, 10 days, 11 days, 12 days, 13 days, 14 days, three weeks, one month, 2 months, 3 months, 4 months, 6 months, 7 months, 8 months, 9 months, 10 months, 11 months, or one or more years (inclusive of all time periods falling in between these values) following the onset of an ischemic event caused by cardiovascular disease or MI.

In some embodiments, a therapeutically effective amount of dosage or concentration of glycoengineered FSTL1 can be about 0.1 mg/kg body weight, about 0.2 mg/kg body weight, about 0.3 mg/kg body weight, about 0.4 mg/kg body weight, about 0.5 mg/kg body weight, about 0.6 mg/kg body weight, about 0.7 mg/kg body weight, about 0.8 mg/kg body weight, about 0.9 mg/kg body weight, about 1.0 mg/kg body weight, about 1.1 mg/kg body weight, about 1.2 mg/kg body weight, about 1.3 mg/kg body weight, about 1.4 mg/kg body weight, about 1.5 mg/kg body weight, about 1.6 mg/kg body weight, about 1.7 mg/kg body weight, about 1.8 mg/kg body weight, about 1.9 mg/kg body weight, about 2.0 mg/kg body weight, about 2.1 mg/kg body weight, about 2.2 mg/kg body weight, about 2.3 mg/kg body weight, about 2.4 mg/kg body weight, about 2.5 mg/kg body weight, about 2.6 mg/kg body weight, about 2.7 mg/kg body weight, about 2.8 mg/kg body weight, about 2.9 mg/kg body weight, about 3.0 mg/kg body weight, about 3.1 mg/kg body weight, about 3.2 mg/kg body weight, about 3.3 mg/kg body weight, about 3.4 mg/kg body weight, about 3.5 mg/kg body weight, about 3.6 mg/kg body weight, about 3.7 mg/kg body weight, about 3.8 mg/kg body weight, about 3.9 mg/kg body weight, about 4.0 mg/kg body weight, about 4.1 mg/kg body weight, about 4.2 mg/kg body weight, about 4.3 mg/kg body weight, about 4.4 mg/kg body weight, about 4.5 mg/kg body weight, about 4.6 mg/kg body weight, about 4.7 mg/kg body weight, about 4.8 mg/kg body weight, about 4.9 mg/kg body weight, about 5.0 mg/kg body weight, about 5.1 mg/kg body weight, about 5.2 mg/kg body weight, about 5.3 mg/kg body weight, about 5.4 mg/kg body weight, about 5.5 mg/kg body weight, about 5.6 mg/kg body weight, about 5.7 mg/kg body weight, about 5.8 mg/kg body weight, about 5.9 mg/kg body weight, about 6.0 mg/kg body weight, about 6.1 mg/kg body weight, about 6.2 mg/kg body weight, about 6.3 mg/kg body weight, about 6.4 mg/kg body weight, or about 6.5 mg/kg body weight. In some embodiments, the glycoengineered FSTL1 can be delivered daily. In some embodiments, a therapeutically effective amount of dosage or concentration of glycoengineered FSTL1 can be any amount in the range between 0.2 and 6.0 mg/kg body weight, delivered daily.

### Effects of Glycoengineered FSTL1

Treating a condition can include alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. A disease or condition, such as a heart failure, can be treated with a therapeutically effective amount of dosage or concentration of a drug, e.g., a glycoengineered FSTL1, effective to treat the disease or condition. The therapeutically effective amount can vary depending, for example, but not limited to, on the compound, the disease or the condition and/or symptoms thereof, severity of the disease or the condition and/or symptoms thereof, the age, weight, and/or health of the subject to be treated, and the judgment of the prescribing physician. An appropriate amount in any given instance can be ascertained by those skilled in the art or capable of determination by routine experimentation.

According to any of the methods described herein, repairing cardiac injury with the glycoengineered FSTL1 of the present disclosure can result in increased recovery of the cardiac tissue. In some embodiments, the repairing can result in a reduction in fibrotic scar area in the cardiac tissue following the injury. In some embodiments, the repairing can result in an increase of perfused area in the cardiac tissue. In some embodiments, the repairing can result in an increase in cardiomyocyte cytokinesis in the cardiac tissue. In some embodiments, the cardiomyocyte cytokinesis can be determined by Aurora B kinase expression. In some embodiments, the repairing can result in an increased number of cardiomyocytes. In some embodiments, the repairing can result in an increased level of transcripts encoding cardiac-specific contractile proteins in the cardiomyocytes. In some embodiments, the cardiac-specific contractile proteins can be myh6, mlc2v, and/or mlc2a. In some embodiments, the repairing results in an increase of actinin⁺ cells with rhythmic contractile Ca²⁺ in cardiomyocytes. In some embodiments, the repairing can result in an increase of cardiomyocyte cell cycle entry. In some embodiments, the cardiomyocyte cell cycle entry can be assessed by expression of phosphor-histone H3. In some embodiments, the repairing can result in an increased vascularization in the area of the injury in the cardiac tissue. In some embodiments, the increased vascularization can be determined by expression of von Willebrand factor (vWF) or smooth muscle actin in blood vessel cells.

In some embodiments, administration of the glycoengineered FSTL1 does not activate Akt-1 signaling activity. In other embodiments of the present disclosure, administration of the glycoengineered FSTL1 does not result in decreased apoptosis of cardiomyocytes. In some embodiments, administration of the glycoengineered FSTL1 has a positive action on the contractility of cardiomyocytes. In some embodiments, administration of the glycoengineered FSTL1 increases the maturation of cardiomyocytes. In some embodiments, administration of the glycoengineered FSTL1 improves vascular function. In some embodiments, administration of the glycoengineered FSTL1 improves circulatory hemodynamics

The injury to the cardiac (e.g., myocardial) tissue can be associated with any number of diseases or conditions known to affect the heart or circulatory system and include, without limitation, coronary heart disease, cardiomyopathy, ischemic heart disease, heart failure, inflammatory heart disease, valvular heart disease and aneurysm. In one embodiment, the injury can be caused by myocardial infarction (MI; such as acute myocardial infarction (AMI)). In another embodiment, the injury can be caused by an ischemic event followed by reperfusion.

Repairing cardiac tissue following an injury can mean any type of action or treatment that decreases, minimizes, or even maintains the level of injury due to cardiovascular disease, myocardial infarction, or other ischemic event. Accordingly, repairing an injury can indicate that the subject's condition is not worsened and can be improved with respect to the injury of concern as compared with the level of injury in the absence of treatment or action described herein to reduce injury.

Repair of injured cardiac (e.g., myocardial) tissue can include increasing the number of cardiomyocytes that can be indirectly measure in the live subject by several methods of imaging (like delayed enhance MRI, DE-MRI) as decreased in myocardial infarct size. See for example: Hendel R C et al, JACC 48(7); 1475-97 and Sardella G et al, JACC 2009; 53(4):309-15. In some embodiments, contacting the cardiac (e.g., myocardial) tissue with the glycoengineered FSTL1 of the present disclosure can result in any of about a 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size. In other embodiments, contacting the cardiac (e.g., myocardial) tissue with the glycoengineered FSTL1 of the present disclosure can result in any of about at least 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size. In other embodiments, contacting the cardiac (e.g., myocardial) tissue with the glycoengineered FSTL1 of the present disclosure can result in any of about at most 2%, 5%, 10%, 15%, 20%, 30%, 40% 50%, 60%, 90%, 100%, or about a 5-10%, 10-20%, 20-30%, 30-40%, 40-50%, 50-60%, 60-70%, 70-80%, 80-90%, 90-100% recovery of lost muscle and reduction of infarct size.

In other embodiments, repair of injured cardiac (e.g., myocardial) tissue can include an improvement in the percent fractional shortening of cardiac (e.g., myocardial) tissue compared to the amount of percent fractional shortening in cardiac (e.g., myocardial) tissue that is not contacted by the glycoengineered FSTL1 of the present disclosure following an injury. In some embodiments, contacting the cardiac (e.g., myocardial) tissue with the glycoengineered FSTL1 of the present disclosure can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% or greater improvement in percent fractional shortening of cardiac (e.g., myocardial) tissue compared to same subject prior treatment, inclusive of all values falling in between these percentages.

In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% improvement in percent fractional shortening of cardiac (e.g., myocardial) tissue compared to same subject prior treatment, inclusive of all values falling in between these percentages.

In some embodiments, contacting the cardiac (e.g., myocardial) tissue with the glycoengineered FSTL1 of the present disclosure can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, 90-100% improvement in percent fractional shortening of cardiac (e.g., myocardial) tissue compared to same subject prior treatment, inclusive of all values falling in between these percentages.

In some embodiments, contacting the cardiac (e.g., myocardial) tissue with the glycoengineered FSTL1 of the present disclosure can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, 90-100% improvement in percent fractional shortening of cardiac (e.g., myocardial) tissue compared to same subject prior treatment, inclusive of all values falling in between these percentages.

In some embodiments of any of the methods disclosed herein, about 1, 2, 3, 4, 5, 6, 7, 8, 9, 10, 20, 30, 40, 50, 60, 70, 80, 90, 100, or more fold increase in the number of cardiomyocytes can be achieved compared to the number of cardiomyocytes in cardiac (e.g., myocardial) tissue that is not contacted by the glycoengineered FSTL1 of the present disclosure following an injury. Assessment of cardiomyocyte replication is routine in the art and can be measured by, for example, by determining the number of α-actinin positive cells in a cardiac (e.g., myocardial) tissue sample from a subject.

Repair of injured cardiac (e.g., myocardial) tissue can also include an increase in the amount of cardiomyocyte cytokinesis compared to the amount of cardiomyocyte cytokinesis in cardiac (e.g., myocardial) tissue that is not contacted by the glycoengineered FSTL1 of the present disclosure following an injury. In some embodiments, contacting the cardiac (e.g., myocardial) tissue with the glycoengineered FSTL1 of the present disclosure can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% or greater improvement the amount of cardiomyocyte cytokinesis.

In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% or greater improvement the amount of cardiomyocyte cytokinesis.

In some embodiments, contacting the cardiac (e.g., myocardial) tissue with the glycoengineered FSTL1 of the present disclosure can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% improvement the amount of cardiomyocyte cytokinesis.

In some embodiments, contacting the cardiac (e.g., myocardial) tissue with the glycoengineered FSTL1 of the present disclosure can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% improvement the amount of cardiomyocyte cytokinesis. Assessment of cardiomyocyte cytokinesis is routine in the art and can be measured by, for example, determining the expression level of Aurora B kinase in a cardiac (e.g., myocardial) tissue sample from a subject. In some embodiments, current methods allow these studies to be performed only post-mortem or after biopsy or after transplantation.

In some embodiments, repair of injured cardiac (e.g., myocardial) tissue can comprise decreased cardiomyocyte apoptosis compared to the amount cardiomyocyte apoptosis in cardiac (e.g., myocardial) tissue that is not contacted by the glycoengineered of the present disclosure following an injury.

In some embodiments, contacting the cardiac (e.g., myocardial) tissue with the glycoengineered FSTL1 of the present disclosure can result in any of about a at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or greater reduction in cardiomyocyte apoptosis in cardiac (e.g., myocardial) tissue, inclusive of all values falling in between these percentages.

In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% reduction in cardiomyocyte apoptosis in cardiac (e.g., myocardial) tissue, inclusive of all values falling in between these percentages.

In some embodiments, contacting the cardiac (e.g., myocardial) tissue with the glycoengineered FSTL1 of the present disclosure can result in any of about a at least 1-100%, 5-95%, 10-90%, 20-80%, 30-70%, 5-10%, 10-20%, 20-30%, 30-40%, 40-50%, 50-60%, 60-70%, 70-80%, 80-90%, 90-100% or greater reduction in cardiomyocyte apoptosis in cardiac (e.g., myocardial) tissue, inclusive of all values falling in between these percentages.

In some embodiments, contacting the cardiac (e.g., myocardial) tissue with the glycoengineered FSTL1 of the present disclosure can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% reduction in cardiomyocyte apoptosis in cardiac (e.g., myocardial) tissue, inclusive of all values falling in between these percentages. Assessment of cardiomyocyte apoptosis is routine (post-mortem or ex-vivo, after heart separation) in the art and can be measured by, for example, TUNEL staining of a cardiac (e.g., myocardial) tissue sample from a subject.

Repair of injured cardiac (e.g., myocardial) tissue can also include increased levels of one or more transcripts encoding cardiac-specific contractile proteins in cardiomyocytes compared to the transcriptional level of these contractile proteins in cardiac (e.g., myocardial) tissue that is not contacted by the glycoengineered of the present disclosure following an injury.

In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100% or greater increased level of one or more transcripts encoding cardiac-specific contractile proteins. In some embodiments, contacting the cardiac (e.g., myocardial) tissue with the glycoengineered FSTL1 of the present disclosure can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% increased level of one or more transcripts encoding cardiac-specific contractile proteins.

In some embodiments, contacting the cardiac (e.g., myocardial) tissue with the glycoengineered FSTL1 of the present disclosure can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% increased level of one or more transcripts encoding cardiac-specific contractile proteins.

In some embodiments, contacting the cardiac (e.g., myocardial) tissue with the glycoengineered FSTL1 of the present disclosure can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% increased level of one or more transcripts encoding cardiac-specific contractile proteins. In some embodiments, the cardiac-specific contractile proteins can be selected from the group consisting of myh6, mlc2v, and mlc2a. Assessment of cardiac-specific contractile protein transcript is routine in the art and can be measured by, for example, Northern blot, Western blot, reverse transcriptase (RT) PCR, FACS analysis, immunohistochemistry, or in situ hybridization.

Repair of injured cardiac (e.g., myocardial) tissue can include increased actinin⁺ cells with rhythmic contractile Ca²⁺ in cardiomyocytes. In some embodiments, contacting the cardiac (e.g., myocardial) tissue with the glycoengineered FSTL1 of the present disclosure can result in any of about 1, 2, 3, 4, 5, 6, 7, 8, 9, 10, 20, 30, 40, 50, 60, 70, 80, 90, 100, or more fold increase in the amount of actinin⁺ cells with rhythmic contractile Ca²⁺ in cardiomyocytes compared to the number of actinin⁺ cells with rhythmic contractile Ca²⁺ in cardiac (e.g., myocardial) tissue that is not contacted by the glycoengineered FSTL1 of the present disclosure following an injury. Assessment of actinin⁺ cells with rhythmic contractile Ca²⁺ in cardiomyocytes is routine in the art (See Example 1, infra).

Any of the methods of treating injuries to cardiac (e.g., myocardial) tissue disclosed herein can result in increased survival in a subject following injury. As used herein, increased survival can include, e.g., at least about a 5% (e.g., at least about 10%, 15%, 20%, 25%, 30%, 40%, 45%, 50%, 60%, 70%, 80%, 90%, 100%, 110%, 120%, 130%, 140%, 150% or more than 200% or greater) increase in the survival of a subject compared to relative survival in subjects who have not been subject to the instantly described methods. Survival time can be measured, e.g., in days, weeks, months, or years. In some embodiments, contacting injured cardiac (e.g., myocardial) tissue with the glycoengineered FSTL1 of the present disclosure in accordance with any of the methods described herein can prolong the survival of subject by at least six months, seven months, eight months, nine months, 10 months, 12 months, 18 months, 24 months, 36 months, or more.

In some embodiments, repair of injured cardiac (e.g., myocardial) tissue can include decreased or attenuated fibrosis in cardiac (e.g., myocardial) tissue compared to the amount of fibrosis in cardiac (e.g., myocardial) tissue that is not contacted by the glycoengineered FSTL1 of the present disclosure following an injury. In some embodiments, contacting the cardiac (e.g., myocardial) tissue with the glycoengineered FSTL1 of the present disclosure can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or greater reduction in fibrosis in cardiac (e.g., myocardial) tissue, inclusive of all values falling in between these percentages. In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% reduction in fibrosis in cardiac (e.g., myocardial) tissue, inclusive of all values falling in between these percentages. In some embodiments, contacting the cardiac (e.g., myocardial) tissue with the glycoengineered FSTL1 of the present disclosure can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% reduction in fibrosis in cardiac (e.g., myocardial) tissue, inclusive of all values falling in between these percentages. Assessment of cardiomyocyte fibrosis is routine in the art and can be measured by DE-MRI, or by histologic examination of cardiac (e.g., myocardial) tissue (post-mortem, or biopsy).

Repair of injured cardiac (e.g., myocardial) tissue can additionally include increased vascularization of the injured region of the cardiac (e.g., myocardial) tissue. In some embodiments, contacting the cardiac (e.g., myocardial) tissue with the glycoengineered FSTL1 of the present disclosure can result in any of about at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, or greater recovery or blood-perfused increase in the amount of vascularization in cardiac (e.g., myocardial) tissue compared to the relative amount of vascularization in cardiac (e.g., myocardial) tissue that is not contacted by the glycoengineered FSTL1 of the present disclosure following an injury.

In some embodiments, contacting the cardiac (e.g., myocardial) tissue with the glycoengineered FSTL1 of the present disclosure can result in any of about at most 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% recovery or blood-perfused increase in the amount of vascularization in cardiac (e.g., myocardial) tissue compared to the relative amount of vascularization in cardiac (e.g., myocardial) tissue that is not contacted by the glycoengineered FSTL1 of the present disclosure following an injury. In some embodiments, contacting the cardiac (e.g., myocardial) tissue with the glycoengineered FSTL1 of the present disclosure can result in any of about at least 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% recovery or blood-perfused increase in the amount of vascularization in cardiac (e.g., myocardial) tissue compared to the relative amount of vascularization in cardiac (e.g., myocardial) tissue that is not contacted by the glycoengineered FSTL1 of the present disclosure following an injury. In some embodiments, contacting the cardiac (e.g., myocardial) tissue with the glycoengineered FSTL1 of the present disclosure can result in any of about at most 1-100%, 5-95%, 10-90%, 20-80%, 30-70%, 5-10%, 10-20%, 20-30%, 30-40%, 40-50%, 50-60%, 60-70%, 70-80%, 80-90%, or 90-100% recovery or blood-perfused increase in the amount of vascularization in cardiac (e.g., myocardial) tissue compared to the relative amount of vascularization in cardiac (e.g., myocardial) tissue that is not contacted by the glycoengineered FSTL1 of the present disclosure following an injury. Assessment of vascularization in cardiac (e.g., myocardial) tissue is routine in the art and may be assessed by measuring the expression of proteins such as von Willebrand factor (vWF) or smooth muscle actin in blood vessel cells.

In further embodiments, repair of injured cardiac (e.g., myocardial) tissue can encompass increased cardiomyocyte cell cycle entry. In some embodiments, contacting the cardiac (e.g., myocardial) tissue with the glycoengineered FSTL1 of the present disclosure can result in any of at least about 1, 2, 3, 4, 5, 6, 7, 8, 9, 10, 20, 30, 40, 50, 60, 70, 80, 90, 100, or more fold increase in the amount of cardiomyocyte cell cycle entry in cardiac (e.g., myocardial) tissue compared to the amount of cardiomyocyte cell cycle entry in cardiac (e.g., myocardial) tissue that is not contacted by the glycoengineered FSTL1 of the present disclosure following an injury. Assessment of cardiomyocyte cell cycle entry in cardiac (e.g., myocardial) tissue is routine in the art and may be assessed by measuring the expression of, for example, phosphor-Histone H3.

### EXAMPLES

These examples are provided for illustrative purposes only and not to limit the scope of the claims provided herein.

### Example 1. Expression and Analysis of Glycoengineered FSTL1

The experiments in this Example were performed to generate a glycoengineered FSTL1.

The FSTL1 sequence (SEQ ID NO: 1) was modified by replacing asparagine (N) at positions 144, 175, and 180 with glutamine (Q). The sequence was subsequently codon optimized using GenSmart™ Codon Optimization (GenScript) for optimal gene expression and translation efficiency in mammalian CHO cells (SEQ ID NO: 6).

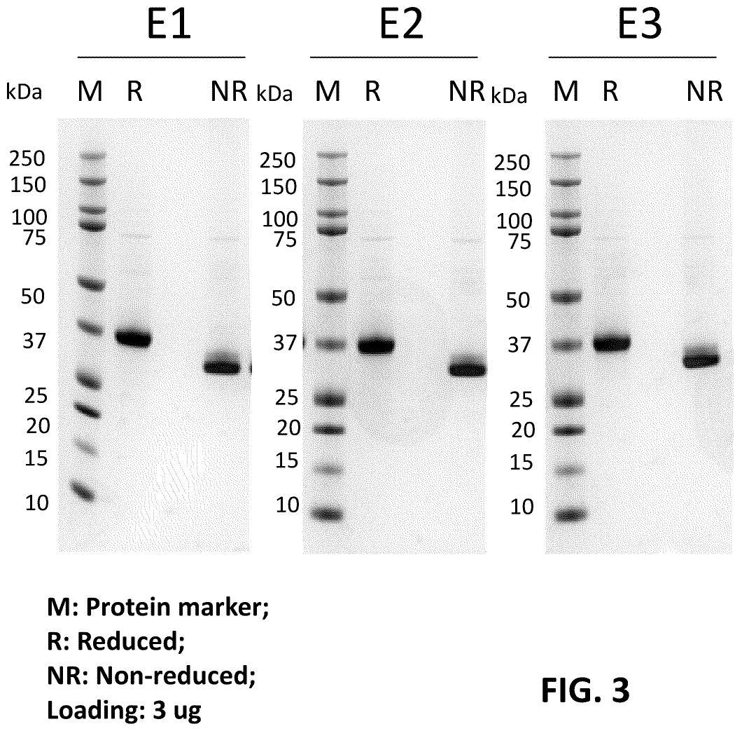

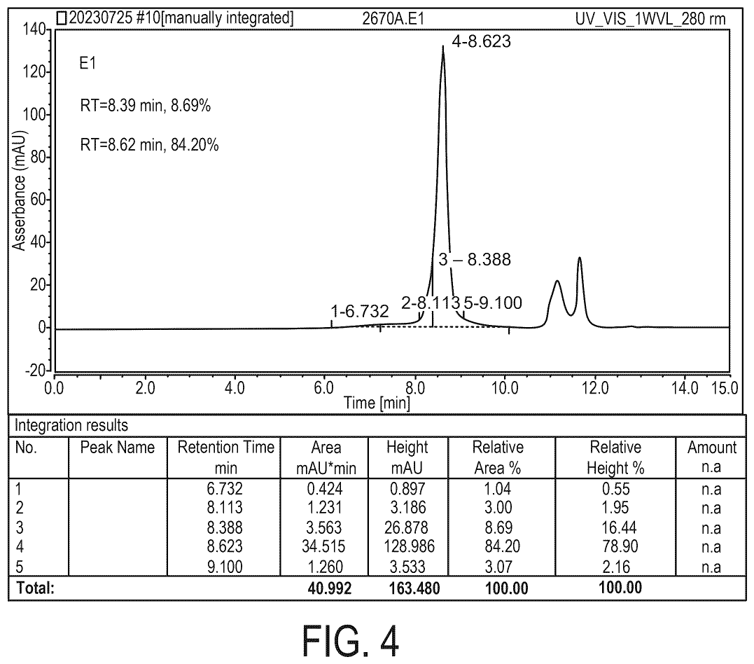

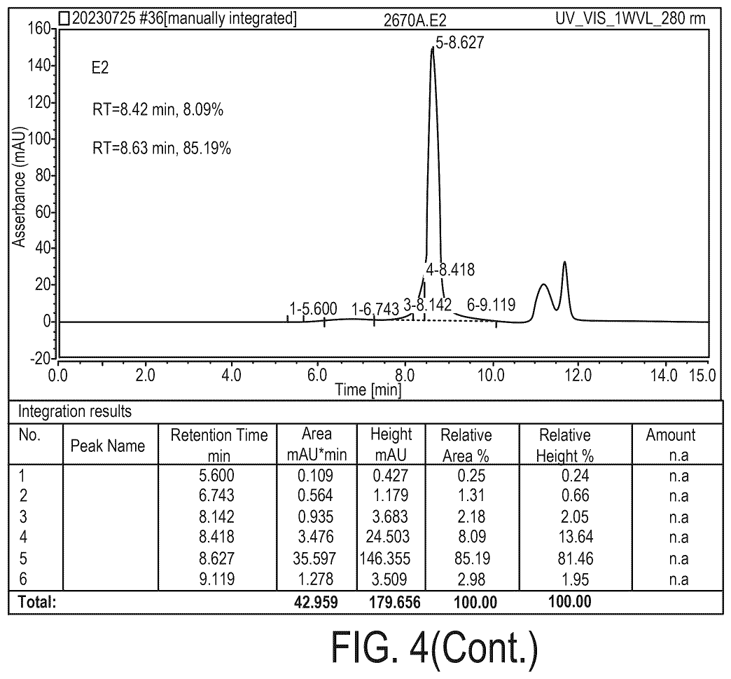

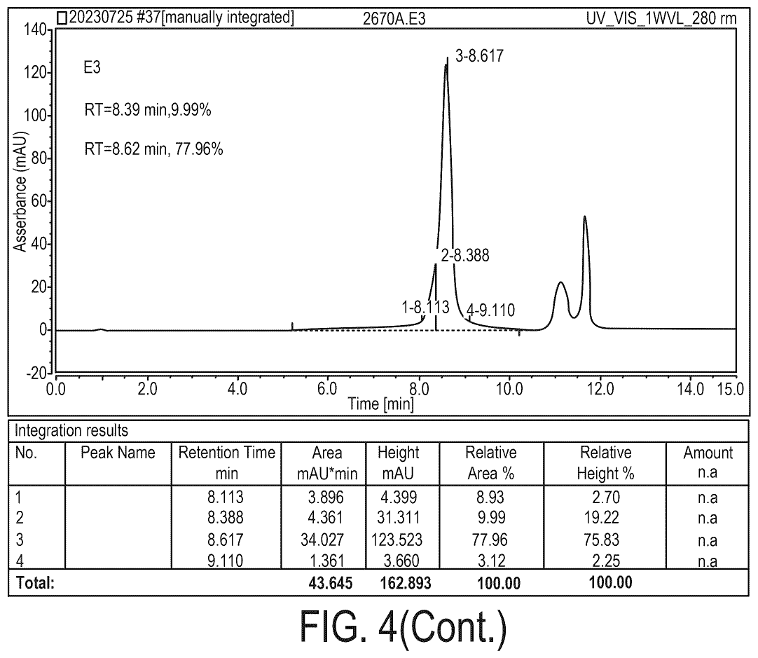

Three different signal peptides (E1, E2, E3) were added to the coding sequence of N-glycosylation depleted FSTL1. The constructs were transiently transfected into CHO cells and evaluated by the following criteria: yield production (SDS/PAGE) (FIG. 3), purity (HPLC) (FIG. 4), efficiency of cleavage (CALIPER) (data not shown), and O-glycosylation content (data not shown). The sequences of the signal peptides were as follows: E1—MGWSCIILFLVATATGVHS (SEQ ID NO: 9), E2—METDTLLLWVLLLWVPGSTG (SEQ ID NO: 10), and E3—MEFGLSWLFLVAILKGVQC (SEQ ID NO: 11). All constructs resulted in similar production and quality. E1 (SEQ ID NO: 9) was selected due to its slightly more efficient yield and cleavage. The obtained optimized sequence (E1) was synthesized and cloned into proper plasmid backbones to obtain the expression vectors. After confirmation of the sequence by sequence analysis, the expression vectors were transfected into CHOk1 cells and stable cell lines were generated with methods well known in the art.

Glycoengineered FSTL1 (SEQ ID NO: 8) was produced by culturing the stable cell lines in 15 L culture over 10-14 days, and collecting the supernatant. The product in supernatants of the fed-batch cultures was purified by Ion Exchange Chromatography (IEX) and Cation Exchange Chromatography (CEX). The glycoengineered FSTL1 was concentrated to 8.3 mg/mL by UFDF and analyzed for product qualities, including SEC-UPLC, reduced and non-reduced SDS Caliper, endotoxin levels, and also Reduced Mass along with characterization of the glycosylation pattern. Analytical data are shown in Table 1. Glycoanalysis is described in Example 2.

TABLE 1
Glycoanalysis of glycoengineered FSTL1

| | SEC- | SDS- | | Measured |
|---|---|---|---|---|
| | HPLC | Caliper | Endotoxin | mass |
| | (% monomer) | NR (%) | (EU/mg) | (Da) |
| Batch 23-10-11 | 92.8% | 96.5 | 0.27 | 33378.5 |

### Example 2. Identification of O-Glycosylation Site on FSTL1

The experiments in this Example were performed to identify O-glycosylation site on FSTL1.

The O-linked glycan is one type of oligosaccharides that links to the hydroxyl group in the side chain of amino acid Serine (S) or Threonine (T) residue, often in S/T-rich domains. As one of the post-translational modifications of bio-therapeutics, it is important to confirm the possible O-glycosylation site and its composition.

The FSTL1 is a single chain glycoengineered protein. According to the peptide mapping results, multiple O-glycosylation were observed, which were annotated as protein with core-1 based O-glycans. To confirm the composition of the O-glycans and to identify the O-glycosylation site, two enzyme digestion, high-resolution tandem mass spectrometry with Electron-Activated Dissociation (EAD) fragmentation were applied to the O-glyco-peptide characterization.

The FSTL1 sample was carried denatured with Guanidine Hydrochloride (Gdn-HCl) in Tris-HCl buffer, reduced by Dithiothreitol (DTT), and followed by cysteine-alkylation with Iodoacetamide (IAM). After sample clean-up, the protein was digested with Lys-C digestion and LysC/trypsin sequential digestion to obtain peptides suitable for subsequent analysis. The sample was injected into an ExionLC coupled with ZenoTOF 7600 mass spectrometer for data collection. The peptides were separated with an Agilent/Poroshell SB-C18 column, and the ultraviolet (UV) chromatograms were set at UV 214 nm. The sample was analyzed with tandem mass spectrometry (MS/MS) fragmentation mode of EAD. High resolution MS and MS/MS spectra were collected. The mass spectra were analyzed by Protein Metrics Inc. (PMI)/Byonic search engine based on the theoretical sequences of the FSTL1 and the O-glycosylation database. The MS/MS spectra regarding to the fragmentation of O-glyco-peptide were further confirmed manually.

O-glyco-Peptide Verification by Lys-C and Lys-C/Trypsin Sequential Digestion with EAD Fragmentation

The information regarding O-glyco-peptides was distilled from the raw data of Lys-C and Lys-C/trypsin sequential digested peptides. MS/MS spectra were collected by EAD mode with data-dependent acquisition including top ten ion scans. By high resolution MS scan, the chemical compositions of the O-glycans on the peptide were identified. The peak area of extracted ion chromatograms (EICs) of O-glyco-peptides were used for the calculation of the ratio for O-glycans.

The details of O-glycans verification are shown in Table 2. The measured masses are in accordance with theoretical ones with mass difference within 10.0 ppm. The O-glycosylation site occupancy ratio was semi-quantitated in the EICs by peak area with equation shown below.

$$Ratio = \frac{EIC {area}_{O - glycopeptide}}{\sum EIC {area}_{O - glycopeptides} + EIC {area}_{naked peptide}}$$

TABLE 2
O-glycosylation of the FSTL1 by Lys-C and Lys-C/trypsin sequential digestion

| | | | | Theoretical | Measured | Differ- | | EIC |
|---|---|---|---|---|---|---|---|---|
| | | | RT | Mass | Mass | ence | | Ratio |
| Digestion | AA No. | Sequence | (min) | (Da) | (Da) | (ppm) | PTMs | (%) |
| Lys-C/ | Amino | SVSPSASPV | 27.9 | 2250.0699 | 2250.0710 | 0.5 | / | / |
| trypsin | acids 83- | VCYQSNRD | | | | | | |
| | 102 of | ELR (SEQ ID | | | | | | |
| | SEQ ID | NO: 3) | | | | | | |
| | NO: 7, | SVSPSASPV | 26.7 | 2615.2021 | 2615.2050 | 1.1 | GalNAc- | 1.9 |
| | (amino | VCYQSNRD | | | | | Gal | |
| | acids 103- | ELR (SEQ ID | 27.3 | 2453.1493 | 2453.1422 | −2.9 | GalNAc | 15.0 |
| | 122 of | NO: 3) | | | | | | |
| | SEQ ID | | 28.2 | 2906.2975 | 2906.3032 | 2.0 | GalNAc- | 59.2 |
| | NO: 1) | | | | | | Gal- | |
| | | | | | | | Neu5AC | |
| | | | 29.3 | 3197.3929 | 3197.3943 | 0.4 | GalNAc- | 4.4 |
| | | | | | | | Gal- | |
| | | | | | | | 2Neu5Ac | |
| Lys-C | Amino | GAQTQTEEE | 17.8 | 2268.0692 | 2268.0732 | 1.8 | / | / |
| | acids 254- | MTRYVQEL | | | | | | |
| | 272 of | QK (SEQ ID | | | | | | |
| | SEQ ID | NO: 4) | | | | | | |
| | NO: 7 | | | | | | | |
| | (amino | GAQTQTEEE | 16.5 | 3215.3923 | 3215.3973 | 1.6 | GalNA | 1.3 |
| | acids 274- | MTRYVQEL | | | | | c-Gal- | |
| | 294 of | QK (SEQ ID | | | | | 2Neu5 | |
| | SEQ ID | NO: 4) | | | | | Ac | |
| | NO: 1) | | | | | | | |

Note:
1. Retention time (RT) is reported by Protein Metrics software.
2. All cysteine containing peptides were alkylated.
3. GalNAc/Gal/Neu5Ac refer to the O-glycan moieties. GalNAc refers to N-acetyl-galactosamine, Gal refers to galactose. Neu5Ac refers to N-acetyl-neuraminic acid.
4. The difference was calculated by the following equation. The results were reported with 1 decimal.
$Difference = \frac{Measured mass - Theoretical mass}{Theoretical mass} \times {10}^{6}$ 5. / refers to not applicable.

The modified peptides with O-glycosylation ratio greater than 1.0% were identified and reported. The MS/MS spectra by EAD demonstrated the identification of the O-glyco-peptide. Peptides 83S-R102 (SEQ ID 7) and 254G-K272 (SEQ ID 7) were identified as the O-glyco-peptide in the FSTL1 molecule. The peptides were observed with the major modification of core-1 based O-glycosylation.

O-Glycosylation Site Identification by Lys-C and Lys-C/Trypsin Sequential Digestion with EAD Fragmentation

Electron-Activated Dissociation (EAD) usually helps maintain labile modifications, owing to the high rate of amide bond cleavage and the moderate amount of excess energy. This helps with the fragmentation of the peptide backbone with the modification still intact, and the determination of sites with O-glycans still attached.

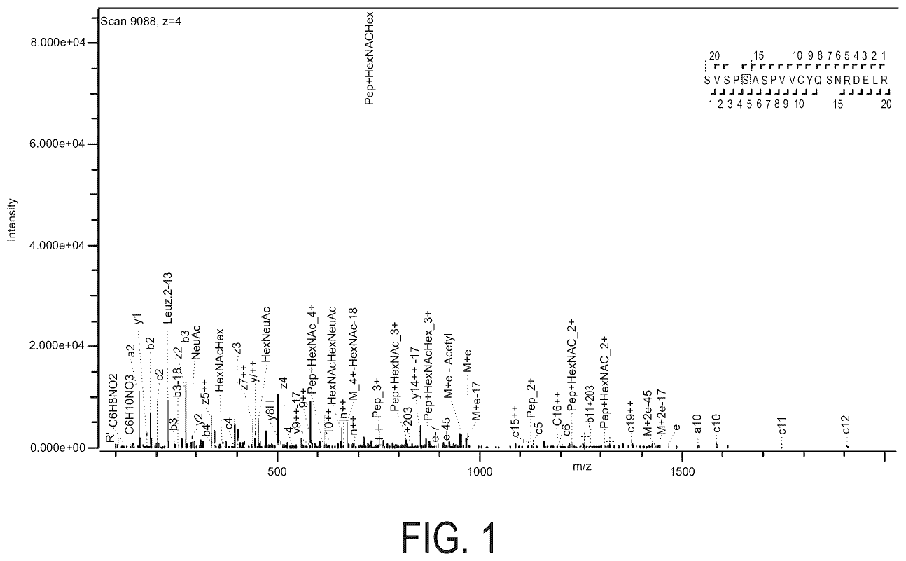

In order to identify the O-glycosylation site, EAD fragmentation technique was applied. The O-glyco-peptide was identified based on the MS and MS/MS data collected by EAD mode with MS/MS scan. The representative MS/MS spectrum of GalNAc-Gal-Neu5Ac modified peptide SC: 83S-R102 from the FSTL1 sample is shown in FIG. 1. The detailed b/y, c/z ions of the modified peptide on SC: 83S-R102 are listed in Table 3.

TABLE 3
Theoretical b/y, c/z ions of GalNAc-Gal-Neu5Ac modified
peptide SC: 83S-R102 of the FSTL1 collected by EAD mode

| No. | b⁺ | b⁺-18 | c⁺ | Sequence | y⁺ | y⁺⁺ | z | No. |
|---|---|---|---|---|---|---|---|---|
| 1 | 88.0393 | 70.0287 | 105.0659 | S | — | — | — | 20 |
| 2 | 187.1077 | 169.0972 | 204.1343 | V | 2820.2727 | 1410.6400 | — | 19 |
| 3 | 274.1397 | 256.1292 | 291.1663 | S | 2721.2043 | 1361.1058 | — | 18 |
| 4 | 371.1925 | 353.1819 | 388.2191 | P | 2634.1723 | 1317.5898 | — | 17 |
| 5 | 1114.4521 | 1096.4416 | 1131.4787 | S | 2537.1195 | 1269.0634 | 2521.1008 | 16 |
| 6 | 1185.4893 | 1167.4787 | 1202.5158 | A | 1793.8599 | 897.4336 | 1777.8412 | 15 |
| 7 | 1272.5213 | 1254.5107 | 1289.5478 | S | 1722.8228 | 861.9150 | 1706.8041 | 14 |
| 8 | 1369.5740 | 1351.5635 | 1386.6006 | P | 1635.7907 | 818.3990 | 1619.7720 | 13 |
| 9 | 1468.6425 | 1450.6319 | 1485.6690 | V | 1538.7380 | 769.8726 | 1522.7193 | 12 |
| 10 | 1567.7109 | 1549.7003 | 1584.7374 | V | 1439.6696 | 720.3384 | 1423.6509 | 11 |
| 11 | 1727.7415 | 1709.7309 | 1744.7681 | C | 1340.6012 | 670.8042 | 1234.5811 | 10 |
| 12 | 1890.8048 | 1872.7943 | 1907.8314 | Y | 1180.5705 | 590.7889 | 1164.5518 | 9 |
| 13 | 2018.8634 | 2000.8529 | 2035.8900 | Q | 1017.5072 | 509.2572 | 1001.4885 | 8 |
| 14 | 2105.8955 | 2087.8849 | 2122.9220 | S | 889.4486 | 445.2279 | 873.4299 | 7 |
| 15 | 2219.9384 | 2201.9278 | 2236.9649 | N | 802.4166 | 401.7119 | 786.3979 | 6 |
| 16 | 2376.0395 | 2358.0289 | 2393.0660 | R | 688.3737 | 344.6905 | 672.3549 | 5 |
| 17 | 2491.0664 | 2473.0559 | 2508.0930 | D | 532.2725 | 266.6399 | 516.2538 | 4 |
| 18 | 2620.1090 | 2602.0985 | 2637.1356 | E | 417.2456 | 209.1264 | 401.2269 | 3 |
| 19 | 2733.1931 | 2715.1825 | 2750.2196 | L | 288.2030 | 144.6051 | 272.1843 | 2 |
| 20 | — | — | — | R | 175.1190 | 88.0631 | 159.1002 | 1 |

Compared to theoretical MS/MS spectra of unmodified peptide SC: 83S-R102, the c4 of GalNAc-Gal-Neu5Ac modified peptide SC: 83S-R102 (from SEQ ID NO: 7) were consistent, while a series of c ions from c5 to c6, c8, and from c10 to c12 shift with 656.2276 Da (corresponding to GalNAc-Gal-Neu5Ac). Combining the MS and MS/MS data together, the 2-glycosylation site of SC: 83-R102 was identified as SC: S87.

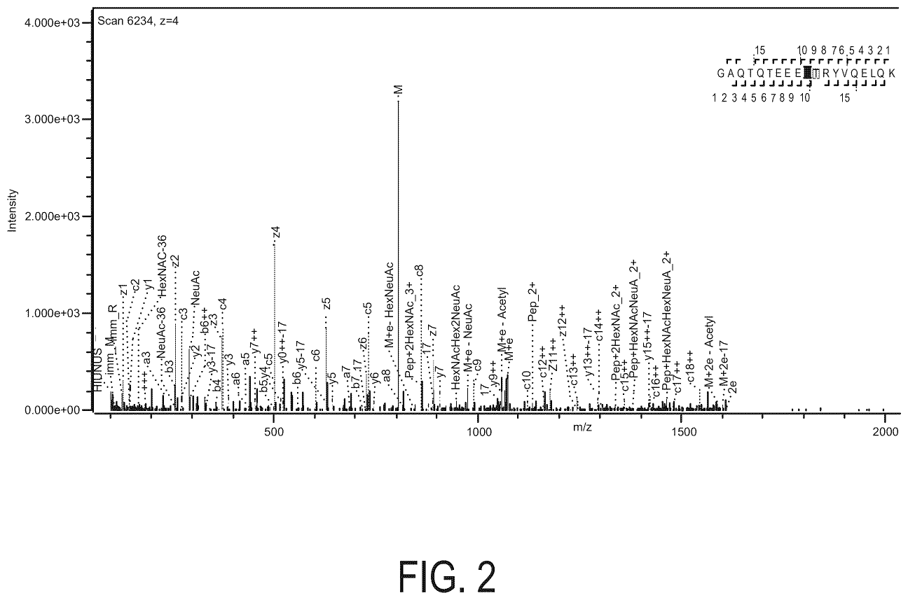

The representative MS/MS spectrum of GalNAc-Gal-2Neu5Ac modified peptide SC: 254G-K272 (from SEQ ID NO: 7) from the FSTL1 sample is shown in FIG. 2. The detailed b/y, c/z ions of the modified peptide on SC: 254G-K272 are listed in Table 4.

TABLE 4
Theoretical b/y, c/z ions of GalNAc-Gal-2Neu5Ac modified
peptide SC: 254G-K272 of the FSTL1 collected by EAD mode

| No. | b⁺ | b⁺-18 | c⁺ | Sequence | y⁺ | y⁺⁺ | z | No. |
|---|---|---|---|---|---|---|---|---|
| 1 | 58.0287 | 40.0182 | 75.0553 | G | — | — | — | 19 |
| 2 | 129.0658 | 111.0553 | 146.0924 | A | 3159.3781 | 1580.1927 | — | 18 |
| 3 | 257.1244 | 239.1139 | 274.1510 | Q | 3088.3409 | 1544.6741 | — | 17 |
| 4 | 358.1721 | 340.1615 | 375.1987 | T | 2960.2824 | 1480.6448 | — | 16 |
| 5 | 486.2307 | 468.2201 | 503.2572 | Q | 2859.2347 | 1430.1210 | — | 15 |
| 6 | 587.2784 | 569.2678 | 604.3049 | T | 2731.1761 | 1366.0917 | — | 14 |
| 7 | 716.3210 | 698.3104 | 733.3475 | E | 2630.1284 | 1315.5678 | — | 13 |
| 8 | 845.3635 | 827.3530 | 862.3901 | E | 2501.0858 | 1251.0466 | — | 12 |
| 9 | 974.4061 | 956.3956 | 991.4327 | E | 2372.0432 | 1186.5253 | — | 11 |
| 10 | 1105.4466 | 1087.4361 | 1122.4732 | M | 2243.0007 | 1122.0040 | 2226.9819 | 10 |
| 11 | 2153.8173 | 2135.8068 | 2170.8439 | T | 2111.9602 | 1056.4837 | 2095.9414 | 9 |
| 12 | 2309.9184 | 2291.9079 | 2326.9450 | R | 1063.5895 | 532.2984 | 1047.5707 | 8 |
| 13 | 2472.9818 | 2454.9712 | 2490.0083 | Y | 907.4884 | 454.2478 | 891.4696 | 7 |
| 14 | 2572.0502 | 2554.0396 | 2589.0767 | V | 744.4250 | 372.7161 | 728.4063 | 6 |
| 15 | 2700.1088 | 2682.0982 | 2717.1353 | Q | 645.3566 | 323.1819 | 629.3379 | 5 |
| 16 | 2829.1513 | 2811.1408 | 2846.1779 | E | 517.2980 | 259.1527 | 501.2793 | 4 |
| 17 | 2942.2354 | 2924.2248 | 2959.2620 | L | 388.2554 | 194.6314 | 372.2367 | 3 |
| 18 | 3070.2940 | 3052.2834 | 3087.3205 | Q | 275.1714 | 138.0893 | 259.1527 | 2 |
| 19 | — | — | — | K | 147.1128 | 74.0600 | 131.0941 | 1 |

Compared to theoretical MS/MS spectra of unmodified peptide SC: 254G-K272, the c10 of GalNAc-Gal-2Neu5Ac modified peptide SC: 254G-K272 were consistent, and there was only one possible O-glycosylation site T264 beyond c10. Combining the MS and MS/MS data together, the O-glycosylation site of SC: 254G-K272 was identified as SC: T7264.

In conclusion, the O-glycosylation site of the FSTL1 was identified as SC: S87 and SC: T264 (SEQ ID NO: 7).

### Example 3. Cardiomyocyte Proliferation by the FSTL1

Advanced LC-MS/MS analysis of conditioned media is utilized as a cornerstone of the methodology. The FSTL1 samples are reduced through the introduction of Tris(2-carboxyethyl)phosphine (TCEP) and subsequently alkylated with iodoacetamide. Mass spectrometry-grade trypsin is then added for digestion, and the resulting peptides are desalted and prepared for analysis.

Utilizing LC-MS/MS system, a comprehensive 120-minute gradient separation of peptides is performed. The LTQ-Orbitrap XL, operating at a resolution of 60,000, is used to scan precursors and subsequently perform data-dependent MS/MS of the top 4 precursors. The raw LC-MS/MS data is processed through Sorcerer Enterprise, facilitating protein identification against the IPI rat protein database, while adhering to stringent search criteria. This includes allowing up to 2 missed cleavages and maintaining a precursor mass tolerance of 50.0 ppm. Furthermore, cysteines are adjusted with a molecular mass of 57 Da for carboxyamidomethylation considerations, with an additional 16 Da for methionine oxidation in the differential search.

Immunohistochemistry analysis in conjunction with histological procedures is carried out, with stringent criteria for the inclusion of FSTL1 engraftment, ensuring the coverage of over 70% of the infarct.

### Example 4. Improvement of Cardiac Function after MI with Localized FSTL1 Delivery

The experiments in this Example are performed to demonstrate that localized FSTL1 delivery improves cardiac function after MI.

Induction of anesthesia is accomplished in mice with 2%, and maintenance is ensured with 1.25-1.5% isoflurane while monitoring the respiratory rate. Subcutaneously inserted ECG leads are used to monitor the heart rate, and body temperature is maintained at 37° C. Functional parameters are recorded using a 3T GE Signa Excite clinical scanner with a dedicated mouse coil. Two crucial sequences are performed for MRI acquisitions. First, DEMRI is executed following IP injection of 0.2 mmol/kg gadopentetate dimeglumine (Magnevist, Berlex Laboratories). Second, cardiac MRI of volumes is performed using fSPGR.

Histological sections of heart samples stained for von Willebrand factor (vWF) are analyzed to measure blood vessel density parameters. Up to 60 sections are analyzed for each treatment group, and ImageJ is used for calculations. Enzyme-linked Immunosorbent Assay is employed to assess FSTL1 retention within the engineered patch system in vitro. Collagen scaffolds laden with FSTL1 (5 μg/ml) are immersed in PBS and shaken for various times. The concentration of FSTL1 is defined using the standard curve of the standard solutions.

In the case of ischemia reperfusion (I/R), a left lateral thoracotomy is performed, and a left anterior descending coronary artery is ligated using an 8-0 Nylon suture against a PE10 tubing. Successful performance of coronary artery occlusion is verified by visual inspection. The chest is then closed using 7-0 sutures, and the skin is closed with 6-0 suture. For the animal group treated with a hydrogel containing the FSTL1, a second thoracotomy is performed one week post the incidence of I/R, and the prepared hydrogel is sutured onto the surface of ischemic myocardium. Sham-operated controls consist of age-matched mice undergoing identical surgical procedures (two thoracotomies) with the exception of LAD ligation. In the ischemia reperfusion study, in vivo heart function is evaluated at baseline, one week after the incidence of I/R, and two and four weeks post-implantation. FSTL1-TG mice used in MI experiments are C57BL6 background, female, and male mice aged 12-15 weeks old.

### Example 5. Activation of Cardiac Regeneration in A Preclinical Swine Model

The experiments in this Example are performed to demonstrate the restorative effect of hydrogel containing FSTL1 delivery in epicardium.

The swine study is conducted through the inflation of a percutaneous coronary angioplasty dilation catheter to occlude the LAD in Yorkshire pigs aged 45 days. Occlusion for 90 minutes is followed by full reperfusion to simulate the clinical MI disease model. One week after MI, a left thoracotomy is performed, and the hydrogel containing the FSTL1 is applied onto the infarct. The animal groups include sham controls, I/R with no treatment, I/R treated with the patch alone, and I/R treated with the patch laden with FSTL1.

EdU delivery is carried out by infusing 250 mg/week EdU into circulation over a 4-week time course of the study, spanning from week 1 to week 5 post I/R using osmotic mini pumps.

Statistical analysis is conducted, with the number of samples (n) recorded. All in vitro experiments are independently performed at least twice, gene expression experiments three times independently, and EdU proliferation assays and cell size measurements more than 10 times independently. Sample sizes for animal studies are estimated, with animals not surviving up to 4 weeks after surgery excluded from functional and histological studies. Randomization is not applied, and blinding to group allocation is practiced between animal surgery and results analysis of mouse myocardial infarction experiments. The values presented are expressed as means t SEM, as SEM quantifies the uncertainty in an estimate of the mean. One-way ANOVA and student T-test are employed to test for statistical significance (P<0.05). Survival curves are generated using PRISM (GraphPad), and the Log-rank (Mantel-Cox) test is used to test the significant differences between the survival of mice in different conditions.

While the disclosure has been particularly shown and described with reference to specific embodiments (some of which are preferred embodiments), it should be understood by those having skill in the art that various changes in form and detail can be made therein without departing from the spirit and scope of the present disclosure as disclosed herein.

### Example 6. Glycoengineered FSTL1 and Non-Glycosylated FSTL1 Size Comparison

The experiments in this Example were performed to demonstrate the comparison of sizes between a glycosylated FSTL1, its non-glycosylated counterpart, and glycoengineered FSTL1 using SDS-PAGE.

Three forms of Follistatin-like 1 (FSTL1) protein were prepared for comparative analysis: non-glycosylated, glycosylated, and glycoengineered. The non-glycosylated FSTL1 was expressed in Escherichia coli (E. coli), while the glycosylated FSTL1 was expressed in a HEK293 mammalian cell line. The glycoengineered FSTL1 was prepared according to the methods described in Example 1 and Example 7.

All protein samples were purified using standard chromatographic techniques, including affinity chromatography and ion-exchange chromatography. The purified proteins were diluted in phosphate-buffered saline (PBS) to a final concentration of 10 ng/μL. To prepare the samples for electrophoresis, 7.5 μL of 4×LDS buffer (pH 8.4, containing lithium dodecyl sulfate) was added to each sample to denature the proteins and reduce disulfide bonds. The samples were then incubated at 70° C. for 10 minutes, followed by immediate cooling on ice.

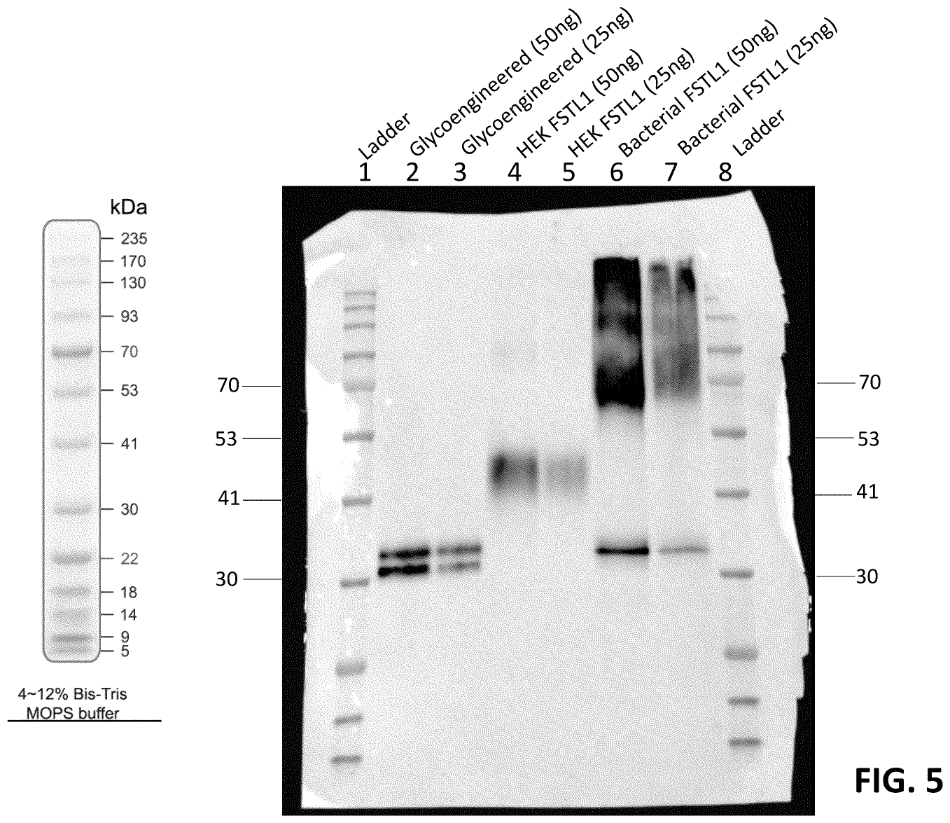

For SDS-PAGE analysis, 2.5 μL of pre-stained protein ladder (extra range molecular weight marker, 5-245 kDa) was loaded into lanes 1 and 8 of a 10% SDS-PAGE gel. 30 μL of each FSTL1 protein sample was loaded into lanes 2 through 7 (FIG. 5).

Electrophoresis was carried out under standard denaturing conditions using 10% polyacrylamide gel, and proteins were separated based on their molecular weights. The inclusion of the molecular weight marker enabled estimation of the apparent molecular weights of the protein bands.

Following electrophoresis, the proteins were transferred onto a polyvinylidene fluoride (PVDF) membrane using a standard wet transfer method. The membrane was then blocked and probed with a primary anti-FSTL1 antibody. Detection was carried out using a horseradish peroxidase (HRP)-conjugated secondary antibody (Goat anti-rabbit IgG).

Bands were visualized using Immobilon Western Chemiluminescent HRP Substrate (Millipore, catalog number WBKLS0500) according to the manufacturer's instructions. The detected bands corresponded to the expected molecular weights of the non-glycosylated, glycosylated, and glycoengineered FSTL1 proteins. As expected, glycoengineered FSTL1 and bacterial FSTL1 proteins migrated faster than HEK FSTL1. The size differences of bacterial FSTL1 and glycoengineered FSTL1 are resolved in Example 7 using mass spectrometry.

### Example 7. Clonal Expansion and Screening of Glycoengineered FSTL1 and Size Comparison of Glycoengineered FSTL1 and Non-Glycosylated FSTL1

The experiments in this Example were performed to demonstrate the systemic selection and evaluation of high-producing, glycoengineered FSTL1 monoclonal CHO clones through single-cell cloning, productivity screening, and comprehensive product quality analysis.

For the cloning of glycoengineered FSTL1, single cells were sorted into each well of 96-well plates using a single cell printer and time-course images were taken using Cell Metric™ (Solentim) imaging system. Thirteen days after single cell cloning, octet screening was performed. The clones with relative higher octet titer and verified monoclonality were transferred to 24-well plates and subsequently expanded into spin tubes. A total of 40 clones were expanded, cryopreserved, and screened by fed-batch study in spin tubes.

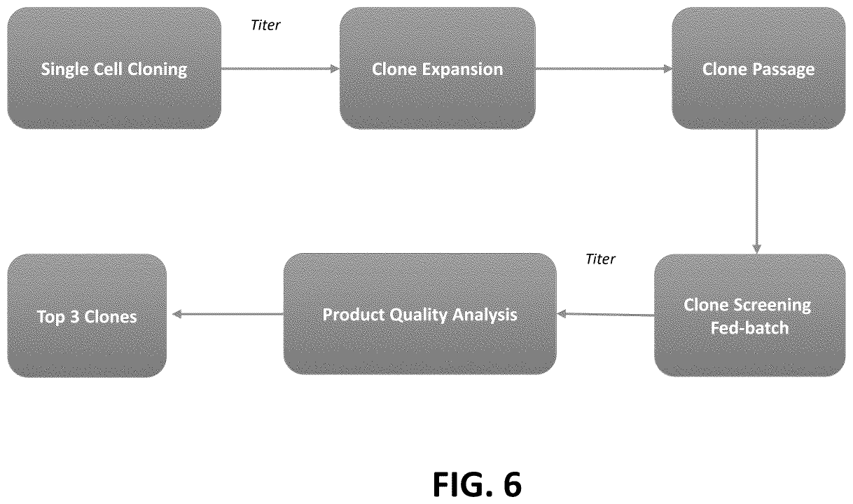

The top 10 clones with relatively higher titer were selected for product quality analysis. The supernatants of the corresponding fed-batch cultures were purified by Ion Exchange Chromatography (IEX) and analyzed for product quality. Based on the assessments of the clonality images, pool diversity, fed-batch growth profile, fed-batch metabolism profile, productivity, and product quality, top 3 clones were selected. FIG. 6 summarizes the workflow involved in cloning and clone screening.

The cloning medium used for single cell cloning was prepared as follows: 79% (v/v) of BM001H+20% (v/v) of host cell spent medium+1% (v/v) of ClonaCell™-CHO ACF Supplement. The host cell spent medium was freshly collected from CHO-K1 host cell cultures by centrifugation at 300×g for 10 min. The prepared cloning medium was filtered by a 0.1-μm filter. Then, 100 μL of the cloning medium was added to each well of the 96-well plates, which were incubated in static CO₂ incubators (36.5° C., 6% CO₂) before use.

On the day of cloning, pool cells were counted by Vi-Cell XR (Beckman Coulter) before sorting. According to cell counting results, required cell volume was calculated and obtained, and then followed by centrifugation at 290×g for 5 min. After that, cell pellets were resuspended with 5 mL CD CHO medium at a final concentration of 1.5×10⁶ cells/mL, and the prepared cell suspension was ready for sorting.

Single cells were sorted into each well of 96-well plates using a single cell printer. Subsequently, cells were incubated in static CO₂ incubators (36.5° C., 6% CO₂). To monitor clone growth, each well of the 96-well plates was imaged by Cell Metric™ (Solentim) imaging system on day 0 (the day of single cell cloning, before and after sorting), day 1 and day 5 after single cell cloning. 90 μL of fresh BM001HB9Z4 medium was added to each well of the 96-well plates on day 7.

Thirteen days after single cell cloning, clones in the 96-well plates were screened for productivity using the Octet method. Two days prior to the assay, cell suspension in each well of the 96-well plates was exchanged with 70 μL of fresh BM001HB4Z2 medium. Supernatant from each well was sampled for the Octet assay. Top 57 clones with relatively higher Octet titer and verified monoclonality were selected and expanded.

The selected clones were expanded from 96-well plates to 24-well plates. 24-well plates were incubated in the static CO₂ incubators (36.5° C., 6% CO₂). After 3 to 4 days, cells were expanded to spin tubes, which were incubated in the shaking incubators (36.5° C., 75% humidity, 6% CO₂, 225 RPM). After being transferred to spin tubes and cultured for 1˜3 days, the clones were then counted and subcultured at 0.3-1.5×10⁵ cells/mL. At this passage, the population doubling levels (PDLs) of the clones were set to zero. Fresh BM001HB4Z2 medium was used for clone expansion and passage. Clones that were unable to be expanded were discarded. Cryopreservation of the clones was performed during cell passage using cryopreservation medium, which consisted of 90% (v/v) BM001H and 10% (v/v) DMSO. The cells were frozen at −80° C. in freezing containers before transferred to a liquid nitrogen tank for long-term storage.

40 clones were evaluated by fed-batch cultures using the protocol shown in the Table 5. The feeding percentage and feeding day were adjusted for some clones based on the growth and metabolism profiles. Glucose level was also maintained throughout the process. All the media used were chemically defined.

TABLE 5
Protocol of clone screening fed-batch cultures

| N-1 media | BM001HB4Z2 | | |
|---|---|---|---|
| Production media | BM020H | | |
| Feed media | FM020a, FM020b (10:1) | | |
| Seeding density | 0.4 × 10⁶ cells/mL | | |
| | Feeding day | FM020a | FM020b |
| Feeding strategy | D 3 | 3% | 0.3% |
| | D 5 | 3% | 0.3% |
| | D 7 | 3% | 0.3% |
| | D 9 | 3% | 0.3% |
| | D 11 | 3% | 0.3% |
| Harvest criteria | D 14 or viability drops below 60% whichever comes first | | |
| Culture conditions | 36.5° C., then temperature shift to 33° C., when VCD reaches about | | |
| | 10.0 × 10⁶ cells/mL (no later than day 6). 75% humidity, 6% CO₂, | | |
| | 225 RPM | | |
| Vessel (working | 50 mL spin tube (20 mL) | | |
| vol.) | | | |

The viability (VIA) and viable cell density (VCD) of clones during the clone screening fed-batch culture were measured using Vi-Cell XR cell viability analyzer. Sampling was performed on each feeding day. The supernatants from harvested cultures were measured for titer by Octet (FSTL1 Antibody, Rabbit MAb). Productivity, growth and metabolism profiles of the top 10 clones in fed-batch cultures are summarized in Table 6.

TABLE 6
Summary of the top 10 clones fed-batch cultures

| | | | Avg. Qp | IVCD | Peak VCD | End VCD | End | End |
|---|---|---|---|---|---|---|---|---|
| | Harvest | End titer | (pg/cell/ | (10⁶ cells/ | (10⁶ cells/ | (10⁶ cells/ | VIA | lactate |
| Sample ID | day | (mg/L) | day) | mL · day) | mL) | mL) | (%) | (g/L) |
| Clone 1-1 | 14 | 3263.6 | 20.4 | 160 | 16.0 | 12.0 | 78.1 | 0.1 |
| Clone 1-3 | 14 | 3530.6 | 22.9 | 154 | 16.1 | 16.1 | 86.7 | 0.1 |
| Clone 1-11 | 14 | 3497.5 | 23.0 | 152 | 15.7 | 13.0 | 82.3 | ND |
| Clone 1-17 | 14 | 4054.4 | 23.6 | 172 | 17.5 | 17.5 | 92.1 | ND |
| Clone 1-18 | 14 | 3843.3 | 25.9 | 148 | 16.7 | 12.9 | 82.7 | ND |
| Clone 1-22 | 14 | 3540.9 | 19.9 | 178 | 17.9 | 15.1 | 74.2 | 0.3 |
| Clone 1-25 | 14 | 3573.9 | 21.7 | 164 | 17.4 | 13.8 | 84.2 | 0.1 |
| Clone 2-15 | 14 | 3521.0 | 22.0 | 160 | 18.9 | 18.9 | 88.1 | ND |
| Clone 2-16 | 14 | 3546.1 | 17.2 | 207 | 25.8 | 17.9 | 92.9 | 0.1 |
| Clone 2-17 | 14 | 3602.8 | 20.8 | 173 | 17.6 | 17.6 | 87.3 | ND |

During the fed-batch study, the top 10 clones exhibited good growth profiles. The peak VCD of top 10 clones were all over 10.0×10⁶ cells/mL. Cell viability was maintained well during the whole fed-batch process, and the end-of-run viabilities of all top 10 clones were over 74%.

The fed-batch cultures supernatants of the top 10 clones were purified by Ion Exchange Chromatography (IEX) and analyzed for product quality, including size exclusion chromatography—ultra performance liquid chromatography (SEC-UPLC) (Table 7), reduced and non-reduced sodium dodecyl sulfate (SDS) caliper (Table 8), capillary isoelectric focusing (cIEF) (Table 9), and mass spectrometry (Table 10).

TABLE 7
SEC-UPLC results of the top 10 clones

| Sample ID | Main peak (%) | HMW peak (%) | LMW peak (%) |
|---|---|---|---|
| Clone 1-1 | 70.2 | 29.0 | 0.8 |
| Clone 1-3 | 64.2 | 35.4 | 0.4 |
| Clone 1-11 | 65.4 | 33.9 | 0.7 |
| Clone 1-17 | 66.6 | 32.5 | 0.9 |
| Clone 1-18 | 67.9 | 31.5 | 0.6 |
| Clone 1-22 | 65.3 | 34.0 | 0.8 |
| Clone 1-25 | 64.0 | 35.4 | 0.6 |
| Clone 2-15 | 62.3 | 37.3 | 0.4 |
| Clone 2-16 | 66.0 | 33.3 | 0.7 |
| Clone 2-17 | 62.6 | 37.0 | 0.4 |

The main peak of the top 10 clones tested by SEC were all over 62%, the overall aggregation level is all lower than 38%, which is acceptable.

TABLE 8
SDS caliper results of the top 10 clones

| | Non-reduced | Reduced |
|---|---|---|
| | SDS caliper | SDS caliper |
| Sample ID | Purity (%) | Purity (%) |
| Clone 1-1 | 73.3 | 91.4 |
| Clone 1-3 | 73.7 | 94.3 |
| Clone 1-11 | 85.9 | 92.6 |
| Clone 1-17 | 77.7 | 92.8 |
| Clone 1-18 | 92.0 | 95.5 |
| Clone 1-22 | 76.5 | 92.8 |
| Clone 1-25 | 74.0 | 92.7 |
| Clone 2-15 | 78.9 | 94.9 |
| Clone 2-16 | 83.0 | 94.1 |
| Clone 2-17 | 80.8 | 95.3 |

The purity of the top 10 clones tested by SDS caliper were all over 73%, which is acceptable.

TABLE 9
cIEF results of the top 10 clones

| | | Acid | Main | Basic |
|---|---|---|---|---|
| Sample ID | pI | peaks (%) | peaks (%) | peaks (%) |
| Clone 1-1 | 6.1 | 51.4 | 27.6 | 20.9 |
| Clone 1-3 | 6.1 | 55.7 | 28.6 | 15.7 |
| Clone 1-11 | 6.1 | 52.5 | 29.9 | 17.6 |
| Clone 1-17 | 6.1 | 56.2 | 28.2 | 15.6 |
| Clone 1-18 | 6.1 | 51.1 | 31.1 | 17.8 |
| Clone 1-22 | 6.1 | 52.2 | 28.2 | 19.6 |
| Clone 1-25 | 6.1 | 56.2 | 29.9 | 13.9 |
| Clone 2-15 | 6.1 | 52.4 | 33.2 | 14.4 |
| Clone 2-16 | 6.1 | 54.9 | 28.7 | 16.4 |
| Clone 2-17 | 6.1 | 54.3 | 30.2 | 15.4 |

The charge variance distribution of the top 10 clones was all acceptable.

TABLE 10
Reduced mass results of the top 10 clones

| | | Expected | Mass |
|---|---|---|---|
| Sample ID | Species | Mass (Da) | (Da) |
| Clone 1-1 | Non-glycosylated | 32720.8 | 32732.5 |
| | Glycoengineered (GalNac) | 32924.0 | 32924.5 |
| | Glycoengineered | 33377.4 | 33378.5 |
| | (GalNAcGalNeu5Ac) | | |
| | Glycoengineered | 33668.7 | 33671.0 |
| | (GalNAcGal2Neu5Ac) | | |
| Clone 1-3 | Non-glycosylated | 32720.8 | 32721.4 |
| | Glycoengineered (GalNac) | 32924.0 | 32924.5 |
| | Glycoengineered | 33377.4 | 33378.5 |
| | (GalNAcGalNeu5Ac) | | |
| | Glycoengineered | 33668.7 | 33671.0 |
| | (GalNAcGal2Neu5Ac) | | |
| Clone 1-11 | Non-glycosylated | 32720.8 | 32721.5 |
| | Glycoengineered (GalNac) | 32924.0 | 32924.5 |
| | Glycoengineered | 33377.4 | 33378.5 |
| | (GalNAcGalNeu5Ac) | | |
| | Glycoengineered | 33668.7 | 33668.7 |
| | (GalNAcGal2Neu5Ac) | | |
| Clone 1-17 | Non-glycosylated | 32720.8 | 32721.6 |
| | Glycoengineered (GalNac) | 32924.0 | 32925.0 |
| | Glycoengineered | 33377.4 | 33379.0 |
| | (GalNAcGalNeu5Ac) | | |
| | Glycoengineered | 33668.7 | 33672.0 |
| | (GalNAcGal2Neu5Ac) | | |
| Clone 1-18 | Non-glycosylated | 32720.8 | 32721.6 |
| | Glycoengineered (GalNac) | 32924.0 | 32925.0 |
| | Glycoengineered | 33377.4 | 33378.5 |
| | (GalNAcGalNeu5Ac) | | |
| | Glycoengineered | 33668.7 | 33671.0 |
| | (GalNAcGal2Neu5Ac) | | |
| Clone 1-22 | Non-glycosylated | 32720.8 | 32721.5 |
| | Glycoengineered (GalNac) | 32924.0 | 32924.5 |
| | Glycoengineered | 33377.4 | 33378.5 |
| | (GalNAcGalNeu5Ac) | | |
| | Glycoengineered | 33668.7 | 33670.5 |
| | (GalNAcGal2Neu5Ac) | | |
| Clone 1-25 | Non-glycosylated | 32720.8 | 32721.0 |
| | Glycoengineered (GalNac) | 32924.0 | 32924.0 |
| | Glycoengineered | 33377.4 | 33378.0 |
| | (GalNAcGalNeu5Ac) | | |
| | Glycoengineered | 33668.7 | 33671.1 |
| | (GalNAcGal2Neu5Ac) | | |
| Clone 2-15 | Non-glycosylated | 32720.8 | 32721.5 |
| | Glycoengineered (GalNac) | 32924.0 | 32925.0 |
| | Glycoengineered | 33377.4 | 33378.5 |
| | (GalNAcGalNeu5Ac) | | |
| | Glycoengineered | 33668.7 | 33671.4 |
| | (GalNAcGal2Neu5Ac) | | |
| Clone 2-16 | Non-glycosylated | 32720.8 | 32721.0 |
| | Glycoengineered (GalNac) | 32924.0 | 32924.5 |
| | Glycoengineered | 33377.4 | 33378.5 |
| | (GalNAcGalNeu5Ac) | | |
| | Glycoengineered | 33668.7 | 33671.0 |
| | (GalNAcGal2Neu5Ac) | | |
| Clone 2-17 | Non-glycosylated | 32720.8 | 32721.5 |
| | Glycoengineered (GalNac) | 32924.0 | 32925.0 |
| | Glycoengineered | 33377.4 | 33378.5 |
| | (GalNAcGalNeu5Ac) | | |
| | Glycoengineered | 33668.7 | 33671.1 |
| | (GalNAcGal2Neu5Ac) | | |

Based on an overall assessment of clone performance and quality including growth and metabolism profiles, productivity and product quality, Clones 1-11, 1-18, and 2-16 were selected as the top 3 clones.

We claim:

1. A glycoengineered follistatin-like 1 (FSTL1) polypeptide, wherein the polypeptide (i) comprises O-linked glycosylation at one or more O-linked glycosylation-competent amino acid residues, and (ii) lacks N-linked glycosylation at one or more N-linked glycosylation-competent amino acid residues.

2. The polypeptide of claim 1, wherein the amino acid sequence of the polypeptide comprises the sequence set forth as SEQ ID NO: 1, SEQ ID NO: 5, SEQ ID NO: 7, or SEQ ID NO: 8 or a fragment or variant thereof having at least 90% sequence identity to the sequence set forth as SEQ ID NO: 1, SEQ ID NO: 5, SEQ ID NO: 7, or SEQ ID NO: 8.

3. The polypeptide of claim 2, wherein
(a) the one or more O-linked glycosylation-competent amino acid residues are at (i) positions 107 and 284 in the sequence set forth as SEQ ID NO: 1; (ii) positions 106 and 283 in the sequence set forth as SEQ ID NO: 5, (iii) positions 87 and 264 in the sequence set forth as SEQ ID NO: 7, or (iv) positions 87 and 264 in the sequence set forth as SEQ ID NO: 8, and
(b) the one or more N-linked glycosylation-competent amino acid residues lacking N-link glycosylation are at (i) positions 144, 175, and 180 in the sequence set forth as SEQ ID NO: 1; (ii) positions 143, 174, and 179 in the sequence set forth as SEQ ID NO: 5; (iii) positions 124, 155, and 160 in the sequence set forth as SEQ ID NO: 7; or (iv) positions 124, 155, and 160 in the sequence set forth as SEQ ID NO: 8.

4. The polypeptide of claim 3, wherein the glycoengineered FSTL1 polypeptide:
(a)(i) comprises O-linked glycosylation at positions 107 and 284 in the sequence set forth as SEQ ID NO: 1, and (ii) lacks N-linked glycosylation at positions 144, 175, and 180 in the sequence set forth as SEQ ID NO: 1;
(b)(i) comprises O-linked glycosylation at positions 106 and 283 in the sequence set forth as SEQ ID NO: 5, and (ii) lacks N-linked glycosylation at positions 143, 174, and 179 in the sequence set forth as SEQ ID NO: 5;
(c)(i) comprises O-linked glycosylation at positions 87 and 264 in the sequence set forth as SEQ ID NO: 7, and (ii) lacks N-linked glycosylation at positions 124, 155, and 160 in the sequence set forth as SEQ ID NO: 7; or
(d)(i) comprises O-linked glycosylation at positions 87 and 264 in the sequence set forth as SEQ ID NO: 7, and (ii) lacks N-linked glycosylation at positions 124, 155, and 160 in the sequence set forth as SEQ ID NO: 8.

5. The polypeptide of claim 2, wherein the one or more N-linked glycosylation-competent amino acid residues in the sequences set forth as SEQ ID NO: 1 and SEQ ID NO: 7 are substituted with a glycosylation-incompetent residue.

6. The polypeptide of claim 5, wherein the glycosylation-incompetent residue is glutamine (Q).

7. The polypeptide of claim 6, wherein the polypeptide comprises the sequence set forth as SEQ ID NO: 5 or SEQ ID NO: 8, or a fragment or variant thereof having at least 90% sequence identity to the sequence set forth as SEQ ID NO: 5 or SEQ ID NO: 8.

8. A nucleic acid encoding the glycoengineered FSTL1 polypeptide of claim 1.

9. The nucleic acid of claim 8, wherein the nucleic acid comprises the sequence set forth as SEQ ID NO: 2 or SEQ ID NO: 6, or a fragment or variant thereof having at least 90% sequence identity to the sequence set forth as SEQ ID NO: 2 or SEQ ID NO: 6.

10. A vector comprising the nucleic acid of claim 8.

11. The vector of claim 10, wherein the vector comprises the nucleic acid sequence set forth as SEQ ID NO: 2 or SEQ ID NO: 6, or a fragment or variant thereof having at least 90% sequence identity to the nucleic acid sequence set forth as SEQ ID NO: 2 or SEQ ID NO: 6.

12. A composition comprising a glycoengineered follistatin-like 1 (FSTL1) polypeptide, wherein the glycoengineered FSTL1 polypeptide (i) comprises O-linked glycosylation at one or more O-linked glycosylation-competent amino acid residues, and (ii) lacks N-linked glycosylation at one or more N-linked glycosylation-competent amino acid residues.

13. The composition of claim 12, wherein the amino acid sequence of the glycoengineered FSTL1 polypeptide comprises the sequence set forth as SEQ ID NO: 1, SEQ ID NO: 5, SEQ ID NO: 7, or SEQ ID NO: 8 or a fragment or variant of any thereof having at least 90% sequence identity to the sequence set forth as SEQ ID NO: 1, SEQ ID NO: 5, SEQ ID NO: 7, or SEQ ID NO: 8.

14. The composition of claim 13, wherein
(a) the one or more O-linked glycosylation-competent amino acid residues are at (i) positions 107 and 284 in the sequence set forth as SEQ ID NO: 1; (ii) positions 106 and 283 in the sequence set forth as SEQ ID NO: 5, (iii) positions 87 and 264 in the sequence set forth as SEQ ID NO: 7, or (iv) positions 87 and 264 in the sequence set forth as SEQ ID NO: 8; and
(b) the one or more N-linked glycosylation-competent amino acid residues lacking N-link glycosylation are at (i) positions 144, 175, and 180 in the sequence set forth as SEQ ID NO: 1; (ii) positions 143, 174, and 179 in the sequence set forth as SEQ ID NO: 5; (iii) positions 124, 155, and 160 in the sequence set forth as SEQ ID NO: 7; or (iv) positions 124, 155, and 160 in the sequence set forth as SEQ ID NO: 8.

15. The composition of claim 14, wherein the glycoengineered FSTL1 polypeptide:
(a)(i) comprises O-linked glycosylation at positions 107 and 284 in the sequence set forth as SEQ ID NO: 1, and (ii) lacks N-linked glycosylation at positions 144, 175, and 180 in the sequence set forth as SEQ ID NO: 1;
(b)(i) comprises O-linked glycosylation at positions 106 and 283 in the sequence set forth as SEQ ID NO: 5, and (ii) lacks N-linked glycosylation at positions 143, 174, and 179 in the sequence set forth as SEQ ID NO: 5;
(c)(i) comprises O-linked glycosylation at positions 87 and 264 in the sequence set forth as SEQ ID NO: 7, and (ii) lacks N-linked glycosylation at positions 124, 155, and 160 in the sequence set forth as SEQ ID NO: 7; or
(d)(i) comprises O-linked glycosylation at positions 87 and 264 in the sequence set forth as SEQ ID NO: 7, and (ii) lacks N-linked glycosylation at positions 124, 155, and 160 in the sequence set forth as SEQ ID NO: 8.

16. The composition of claim 13, wherein the one or more N-linked glycosylation-competent amino acid residues in the sequence set forth as SEQ ID NO: 1 and SEQ ID NO: 7 are substituted with a glycosylation-incompetent residue.

17. The composition of claim 16, wherein the glycosylation-incompetent residue is glutamine (Q).

18. The composition of claim 17, wherein the glycoengineered FSTL1 polypeptide comprises the sequence set forth as SEQ ID NO: 5 or SEQ ID NO: 8, or a fragment or variant of any thereof having at least 90% sequence identity to the sequence set forth as SEQ ID NO: 5 or SEQ ID NO: 8.

19. A pharmaceutical composition comprising the glycoengineered FSTL1 polypeptide of claim 1 and one or more pharmaceutically acceptable excipients.

20. A kit comprising
(a) the pharmaceutical composition of claim 19;
(b) one or more pharmaceutically acceptable excipients; and
(c) an instruction manual for use of the kit.

21. A method of repairing cardiac tissue following an injury in a subject in need thereof, the method comprising contacting the cardiac tissue with a composition comprising a glycoengineered FSTL1 polypeptide.

22. The method of claim 21, wherein the amino acid sequence of the glycoengineered FSTL1 polypeptide comprises the sequence set forth as SEQ ID NO: 1, SEQ ID NO: 5, SEQ ID NO: 7, or SEQ ID NO: 8 or a fragment or variant thereof having at least 90% sequence identity to the sequence set forth as SEQ ID NO: 1, SEQ ID NO: 5, SEQ ID NO: 7, or SEQ ID NO: 8.

23. The method of claim 21, wherein
(a) the one or more O-linked glycosylation-competent amino acid residues are at (i) positions 107 and 284 in the sequence set forth as SEQ ID NO: 1; or (ii) positions 87 and 264 in the sequence set forth as SEQ ID NO: 7; and
(b) the one or more N-linked glycosylation-competent amino acid residues lacking N-link glycosylation are at (i) positions 107 and 284 in the sequence set forth as SEQ ID NO: 1; (ii) positions 106 and 283 in the sequence set forth as SEQ ID NO: 5, (iii) positions 87 and 264 in the sequence set forth as SEQ ID NO: 7, or (iv) positions 87 and 264 in the sequence set forth as SEQ ID NO: 8.

24. The method of claim 23, wherein the glycoengineered FSTL1 polypeptide:
(a)(i) comprises O-linked glycosylation at positions 107 and 284 in the polypeptide sequence of SEQ ID NO: 1, and (ii) lacks N-linked glycosylation at positions 144, 175, and 180 in the polypeptide sequence of SEQ ID NO: 1;
(b)(i) comprises O-linked glycosylation at positions 106 and 283 in the polypeptide sequence of SEQ ID NO: 5, and (ii) lacks N-linked glycosylation at positions 143, 174, and 179 in the polypeptide sequence of SEQ ID NO: 5;
(c)(i) comprises O-linked glycosylation at positions 87 and 264 in the polypeptide sequence of SEQ ID NO: 7, and (ii) lacks N-linked glycosylation at positions 124, 155, and 160 in the polypeptide sequence of SEQ ID NO: 7; or
(d)(i) comprises O-linked glycosylation at positions 87 and 264 in the polypeptide sequence of SEQ ID NO: 7, and (ii) lacks N-linked glycosylation at positions 124, 155, and 160 in the polypeptide sequence of SEQ ID NO: 8.

25. The method of claim 22, wherein one or more N-linked glycosylation-competent amino acid residues in the sequence set forth as SEQ ID NO: 1 and SEQ ID NO: 7 are substituted with a glycosylation-incompetent residue.

26. The method of claim 25, wherein the glycoengineered FSTL1 polypeptide comprises the sequence set forth as SEQ ID NO: 5 or SEQ ID NO: 8, or a fragment or variant thereof having at least 90% sequence identity to the sequence set forth as SEQ ID NO: 5 or SEQ ID NO: 8.

27. The method of claim 21, wherein the repairing results in
(a) increased recovery of the cardiac tissue;
(b) a reduction in fibrotic scar area in the cardiac tissue following the injury;
(c) an increase of perfused area in the cardiac tissue;
(d) an increase in cardiomyocyte cytokinesis in the cardiac tissue;
(e) an increased number of cardiomyocytes;
(f) an increased level of transcripts encoding cardiac-specific contractile proteins in the cardiomyocytes;
(g) an increase of actinin⁺ cells with rhythmic contractile Ca²⁺ in cardiomyocytes;
(h) an increase of cardiomyocyte cell cycle entry; and/or
(i) an increased vascularization in the area of the injury in the cardiac tissue.

28. The method of claim 21, wherein the contacting is immediately following the injury or any time after the injury.

29. The method of claim 21, wherein the composition is delivered to the injury
(a) systemically;
(b) via subcutaneous injection;
(c) via intravenous injection;
(d) endocardially;
(e) epicardially;
(f) by a direct injection;
(g) via a drug-eluting stent;
(h) via a catheter;
(i) via coronary infusion; or
(j) in a hydrogel.

30. The method of claim 21, wherein the glycoengineered FSTL1 polypeptide is expressed at the injury site by use of modifiedRNAs (modRNAs) or genomic editing.


SEQUENCE LISTING (11 sequences)

SEQ ID NO: 1 - protein, 308 aa, Homo sapiens
  REGION 1..308: note = Human FSTL1 amino acid sequence (with signal peptide)
  REGION 1..20: note = signal peptide
MWKRWLALALALVAVAWVRAEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 2 - DNA, 924 bp, Homo sapiens
  source 1..924: mol_type = other DNA
  misc_feature 1..924: note = FSTL1 nucleic acid sequence (encodes SEQ ID NO: 1)
atgtggaaacgctggctcgcgctcgcgctcgcgctggtggcggtcgcctgggtccgcgccgaggaagagctaaggagcaaatccaagatctgtgccaatgtgttttgtggagccggccgggaatgtgcagtcacagagaaaggggaacccacctgtctctgcattgagcaatgcaaacctcacaagaggcctgtgtgtggcagtaatggcaagacctacctcaaccactgtgaactgcatcgagatgcctgcctcactggatccaaaatccaggttgattacgatggacactgcaaagagaagaaatccgtaagtccatctgccagcccagttgtttgctatcagtccaaccgtgatgagctccgacgtcgcatcatccagtggctggaagctgagatcattccagatggctggttctctaaaggcagcaactacagtgaaatcctagacaagtattttaagaactttgataatggtgattctcgcctggactccagtgaattcctgaagtttgtggaacagaatgaaactgccatcaatattacaacgtatccagaccaggagaacaacaagttgcttaggggactctgtgttgatgctctcattgaactgtctgatgaaaatgctgattggaaactcagcttccaagagtttctcaagtgcctcaacccatctttcaaccctcctgagaagaagtgtgccctggaggatgaaacgtatgcagatggagctgagaccgaggtggactgtaaccgctgtgtctgtgcctgtggaaattgggtctgtacagccatgacctgtgacggaaagaatcagaagggggcccagacccagacagaggaggagatgaccagatatgtccaggagctccaaaagcatcaggaaacagctgaaaagaccaagagagtgagcaccaaagagatc

SEQ ID NO: 3 - protein, 20 aa, Homo sapiens
  REGION 1..20: note = FSTL1 amino acid sequence 103-122 of SEQ ID NO: 1
SVSPSASPVVCYQSNRDELR

SEQ ID NO: 4 - protein, 19 aa, Homo sapiens
  REGION 1..19: note = FSTL1 amino acid sequence 274-292 of SEQ ID NO: 1
GAQTQTEEEMTRYVQELQK

SEQ ID NO: 5 - protein, 307 aa, synthetic construct
  REGION 1..307: note = Glyco-engineered FSTL1 amino acid sequence (with signal peptide)
  REGION 1..19: note = E1 signal peptide (SEQ ID NO: 9)
MGWSCIILFLVATATGVHSEEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSQYSEILDKYFKNFDNGDSRLDSSEFLKFVEQQETAIQITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 6 - DNA, 921 bp, synthetic construct
  source 1..921: mol_type = other DNA
  misc_feature 1..921: note = Glyco-engineered FSTL1 nucleic acid sequence (encodes SEQ ID NO: 5)
atgggatggtcatgtatcatcctttttctggtagcaactgcaactggagtacatagcgaggaagaactgaggtctaagtccaagatctgtgctaatgttttctgtggcgccggccgcgaatgcgccgtgactgagaaaggcgagccaacatgcctctgcattgagcagtgcaaaccacataagcgccctgtgtgcggcagcaatggaaagacttacctgaaccactgcgagctgcatcgggatgcttgtctgactgggagcaagatccaagtggactacgacggacactgcaaggaaaagaagagtgtcagcccaagcgccagtcccgtggtctgctaccaaagcaatcgggatgagctaaggaggcggatcatacaatggctggaggccgagataattcctgacggctggttctccaaaggtagtcaatattcggaaatcttggacaagtacttcaagaactttgacaacggtgactctcggctggactcttccgagttcctcaagttcgtggaacagcaggaaaccgccatccaaatcactacctatccggaccaagaaaacaacaagctccttcggggcctctgtgtcgatgccctgatcgagctttctgacgagaatgctgactggaaactctcctttcaagaattcctgaagtgtttgaatccttcctttaaccctcctgagaagaaatgcgccctggaggatgagacctacgccgacggggctgagaccgaagttgactgcaatcgctgcgtctgcgcctgtggaaactgggtctgcacggcaatgacctgtgacgggaagaatcagaagggagcacagacacagaccgaggaggaaatgactagatacgtccaagagctgcagaagcaccaggagactgctgagaaaactaagcgcgtgtctaccaaggagatt

SEQ ID NO: 7 - protein, 288 aa, Homo sapiens
  REGION 1..288: note = Human FSTL1 amino acid sequence (mature - without signal peptide)
EEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 8 - protein, 288 aa, synthetic construct
  REGION 1..288: note = Glyco-engineered FSTL1 amino acid sequence (mature - without signal peptide)
EEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSQYSEILDKYFKNFDNGDSRLDSSEFLKFVEQQETAIQITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 9 - protein, 19 aa, synthetic construct
  REGION 1..19: note = E1 signal peptide
MGWSCIILFLVATATGVHS

SEQ ID NO: 10 - protein, 20 aa, synthetic construct
  REGION 1..20: note = E2 signal peptide
METDTLLLWVLLLWVPGSTG

SEQ ID NO: 11 - protein, 19 aa, synthetic construct
  REGION 1..19: note = E3 signal peptide
MEFGLSWLFLVAILKGVQC